(19) United States
(12) Patent Application Publication
(10) Pub. No.: US 2011/0144510 A1
(43) Pub. Date: Jun. 16, 2011

(54) METHODS TO IDENTIFY DAMAGED OR SCARRED TISSUE BASED ON POSITION INFORMATION AND PHYSIOLOGICAL INFORMATION

(75) Inventors: Kyungmoo Ryu, Palmdale, CA (US); Euljoon Park, Valencia, CA (US); Stuart Rosenberg, Castaic, CA (US); Allen Keel, San Francisco, CA (US); Wenbo Hou, Lancaster, CA (US); Thao Thu Nguyen, Bloomington, MN (US); Steve Koh, South Pasadena, CA (US); Kjell Norén, Solna (SE); Michael Yang, Thousand Oaks, CA (US)

(73) Assignee: PACESETTER, INC., Sylmar, CA (US)

(21) Appl. No.: 12/639,788

(22) Filed: Dec. 16, 2009

(51) Int. Cl. A61B 5/0452

(52) U.S. Cl. 600/509

(57) ABSTRACT

An exemplary system includes one or more processors; memory; and control logic, of one or more modules operable in conjunction with the one or more processors and the memory, to acquire myocardial potential data associated with position information, acquire myocardial electrical activation data associated with position information, acquire myocardial position data with respect to time, generate isopotential contours based on the potential data, generate isochronal contours based on the electrical activation data, generate isomotion contours based on the position data with respect to time, and overlay the generated isopotential contours, isochronal contours and isomotion contours on a display to indicate a region of myocardial damage or myocardial scarring with respect to a map that comprises anatomical markers. Various other methods, devices, systems, etc., are also disclosed.

### TECHNICAL FIELD

Subject matter presented herein relates generally to techniques for assessing health of cardiac tissue and cardiac activity for use in cardiac pacing and/or stimulation therapy, cardiac tissue ablation therapy and the like. Various examples include visual mapping of metrics. Such metrics may be based on a combination of position information and physiological information.

### BACKGROUND

Cardiac arrhythmias are a leading cause of death and disability, with more than 250,000 cases of sudden cardiac death (SCD) annually in the United States alone. Studies suggest that approximately 70% of SCD is related to either scar-related monomorphic ventricular tachycardia (VT) or acute infarction/ischemia causing polymorphic VT/ventricular fibrillation (VF). Further, according to, for example, Wilber et al. (Am Heart J 1985; 109:8-18), the extent of myocardial scar is highly related to inducibility of VT. Yet further, according to, for example, Bello et al. (JACC 2005; 45:1104-1108), infarct size might be a better predictor for SCD than ejection fraction.

In many instances, implantable cardiac defibrillators (ICDs) are indicated for patients at risk of SCD (e.g., secondary prevention: patients resuscitated from VT/VF; primary prevention: high risk patients who have not yet had VT/VF). An accurate estimation of infarct size characterization or the extent of myocardial scar or transmural infarct scar may potentially lead to earlier identification or better identification of patients with higher risk for SCD. Given such identification techniques, it may be possible to elaborate and test specific indications as treatable via an ICD, which may further lead to advancements in ICD technology, improved quality of life and associated decreases in healthcare costs.

The DETERMINE (Defibrillators To Reduce Risk by Magnetic Resonance Imaging Evaluation) study, coordinated by researchers at Northwestern University and sponsored by St. Jude Medical, is examining patients who have had a heart attack (myocardial infarction), but whose hearts are generally less damaged, to determine if an ICD therapy may possibly prolong life. The DETERMINE study aims to bridge a gap that exists under current guidelines. Specifically, current guidelines require that physicians use ejection fraction to determine if patients qualify for ICD therapy. At present, patients with a low ejection fraction (e.g., less than 35 percent) qualify for ICD therapy. However, most people who suffer cardiac arrest have an ejection fraction greater than the standard low criterion and therefore are not eligible for the ICD therapy.

While the aforementioned study of Bello et al. states that scar tissue in the lower chamber of the heart, developed after a heart attack, may be an indicator of SCD risk, these studies rely on expensive imaging techniques, specifically Delayed Enhancement Magnetic Resonance Imaging (DE-MRI).

A DE-MRI study requires injection of a contrast agent (e.g., consider gadolinium-based, heavy metal contrast agents) and data acquisition about 10 minutes thereafter. A typically patient study requires about 45 minutes of in scanner time with continuous ECG and blood pressure monitoring. MR data acquisition typically relies on T1 relaxation time weighted ultrafast gradient echo or steady state gradient echo sequences. The contrast of these sequences may be optimized by various techniques (e.g., inversion-recuperation with nulling or Phase Sensitive Inversion Recovery).

DE-MRI has proven to be more sensitive than single photon emission tomography (SPECT) at detection of subendocardial infarcts. DE-MRI can distinguish between acute infarcts with necrotic myocytes and acute infarcts with necrotic myocytes and damaged microvasculature. The latter, termed “no-reflow phenomenon”, indicates compromised tissue perfusion despite restoration of epicardial artery patency (see, e.g., Klem, “CMR Delayed Enhanced Imaging in Coronary Artery Disease,” MAGNETOM Flash, 2/2007). In the article by Klem, long axis MR images were acquired for a patient before and two months after revascularization noting that, even though an akinetic anterior wall was thinned (a diastolic wall thickness of 5 mm versus a remote zone wall thickness of 9 mm), the applied DE-MRI technique could identify a quite thin subendocardial infarction (1.5 mm thick) in the anterior wall. The article of Klem notes that a direct assessment of viability would likely show the anterior wall as being predominantly viable (e.g., anterior wall thickness divided by the sum of anterior wall thickness and infarct thickness or 3.5 mm/5 mm to arrive at 70% viable); whereas, an indirect assessment would show the anterior wall as being predominantly nonviable (e.g., anterior wall thickness divided by remote region wall thickness or 3.5 mm/9 mm to arrive at 39% viable). The article of Klem further notes that cine MR images obtained following coronary revascularization demonstrated full recovery of wall motion and diastolic wall thickness. In general, DE-MRI studies have successfully signified ischemic edema (myocardial infarction in the acute phase), inflammatory or infectious pathology (myocarditis), fibrous reorganization (sequelae of infarct, cardiomyopathies), and tumorous lesion.

While DE-MRI is often used in a clinical setting for characterization of myocardial tissue viability, it is a separate/added procedure with its own additional risks, and is expensive and time consuming to both patient and physician. As described herein, various exemplary approaches can map myocardial characteristics such as scars in real-time and optionally during implant of a cardiac therapy device, an EP procedure, etc.

### SUMMARY

An exemplary system includes one or more processors; memory; and control logic, of one or more modules operable in conjunction with the one or more processors and the memory, to acquire myocardial potential data associated with position information, acquire myocardial electrical activation data associated with position information, acquire myocardial position data with respect to time, generate isopotential contours based on the potential data, generate isochronal contours based on the electrical activation data, generate isomotion contours based on the position data with respect to time, and overlay the generated isopotential contours, isochronal contours and isomotion contours on a display to indicate a region of myocardial damage or myocardial scarring with respect to a map that comprises anatomical markers. Various other methods, devices, systems, etc., are also disclosed.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with at least three leads implanted into a patient's heart and at least one other lead for sensing and/or delivering stimulation and/or shock therapy. Other devices with more or fewer leads may also be suitable.

FIG. 2 is a functional block diagram of an exemplary implantable stimulation device illustrating basic elements that are configured to provide cardioversion, defibrillation, pacing stimulation and/or other tissue stimulation. The implantable stimulation device is further configured to sense information and administer therapy responsive to such information.

FIG. 3 is a block diagram of an exemplary method for optimizing therapy and/or monitoring conditions based at least in part on position and/or physiological information.

FIG. 4 is a block diagram of the exemplary method of FIG. 3 along with various options.

FIG. 5 is an exemplary arrangement of a lead and electrodes for acquiring position information and optionally other information.

FIG. 6 is a simplified diagram illustrating the heart with right ventricular lead electrodes adjacent a septal wall and left ventricular lead electrodes adjacent a lateral wall along with contours that indicate various regions with respect to scar tissue as well as a plot of lead displacement and various electrograms.

FIG. 7 is a simplified diagram illustrating an exemplary mapping method where a composite map includes contours for various metrics and a scar map indicates location of a scar based on the various metrics.

FIG. 8 is a block diagram of an exemplary method for indicating a damaged or scarred region on a map of the heart.

FIG. 9 is a block diagram of an exemplary mapping method and a system configured to display a map of the heart and contours of various metrics that may be adjusted to more accurately indicate a damaged or scarred region.

FIG. 10 is a diagram of an exemplary catheter that may be used for acquiring position information and/or physiological information.

FIG. 11 is an exemplary system for acquiring information and analyzing such information.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators are generally used to reference like parts or elements throughout.

### Overview

As described herein, various exemplary techniques include mapping position information and physiological information to identify myocardial deficiencies such as ischemia and scarring. A localization system provides position information which may be analyzed to determine, for example, motion, extent of motion and timing of motion. A localization system can also provide position information to locate electrodes or sensors used to acquire physiological information. So called composite maps may be analyzed and displayed to allow a clinician to readily identify regions of interest that may affect cardiac performance.

As described herein, various exemplary methods can generate maps to assist placement of leads, electrodes, sensors, etc. For example, a composite map that identifies ischemic or scarred regions of the myocardium can help optimize placement or selection of leads and electrodes for delivery of CRT. Further, information contained in an exemplary composite map can assist in optimization of parameters for delivery of a therapy. In another example, a composite map that identifies regions associated with arrhythmia can help guide ablation therapy to treat or prevent arrhythmia.

### Exemplary Stimulation Device

Various techniques described below may be implemented in connection with a stimulation device that is configured or configurable to delivery cardiac therapy and/or sense information germane to cardiac therapy.

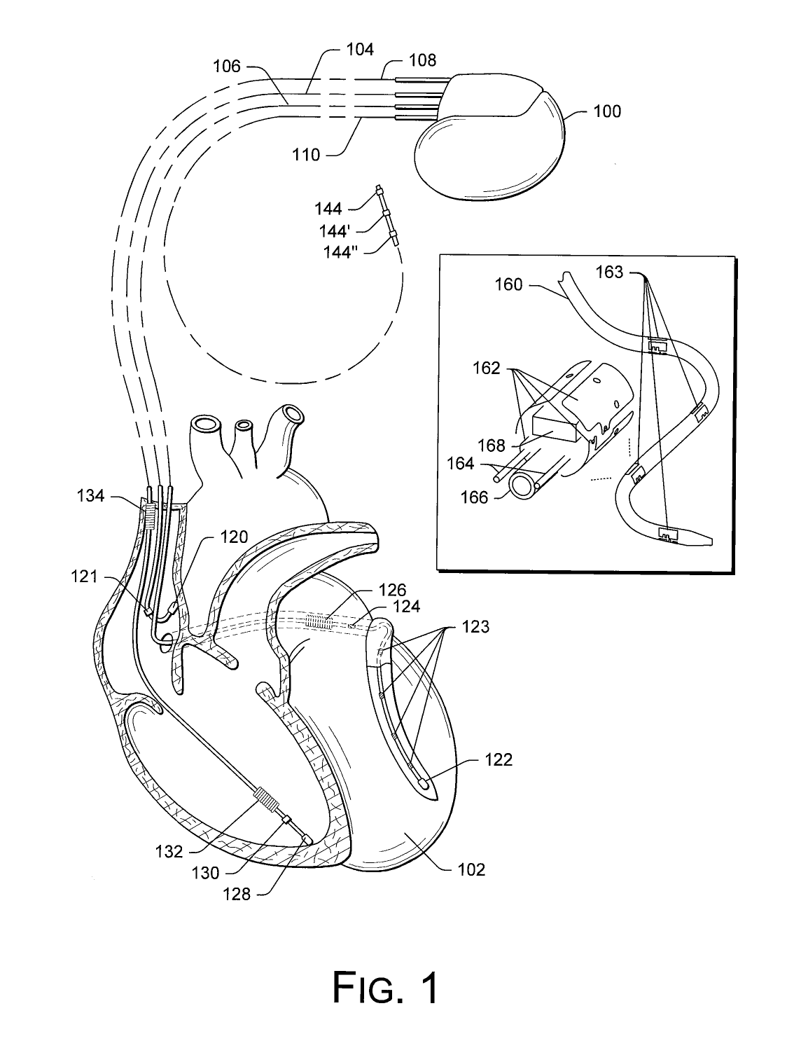

FIG. 1 shows an exemplary stimulation device 100 in electrical communication with a patient's heart 102 by way of three leads 104, 106, 108, suitable for delivering multi-chamber stimulation and shock therapy. The leads 104, 106, 108 are optionally configurable for delivery of stimulation pulses suitable for stimulation of nerves or other tissue. In addition, the device 100 includes a fourth lead 110 having, in this implementation, three electrodes 144, 144′, 144″ suitable for stimulation and/or sensing of physiologic signals. This lead may be positioned in and/or near a patient's heart and/or remote from the heart.

The right atrial lead 104, as the name implies, is positioned in and/or passes through a patient's right atrium. The right atrial lead 104 optionally senses atrial cardiac signals and/or provide right atrial chamber stimulation therapy. As shown in FIG. 1, the stimulation device 100 is coupled to an implantable right atrial lead 104 having, for example, an atrial tip electrode 120, which typically is implanted in the patient's right atrial appendage. The lead 104, as shown in FIG. 1, also includes an atrial ring electrode 121. Of course, the lead 104 may have other electrodes as well. For example, the right atrial lead optionally includes a distal bifurcation having electrodes suitable for stimulation and/or sensing.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or tributary veins of the coronary sinus. Thus, the coronary sinus lead 106 is optionally suitable for positioning at least one distal electrode adjacent to the left ventricle and/or additional electrode(s) adjacent to the left atrium. In a normal heart, tributary veins of the coronary sinus include, but may not be limited to, the great cardiac vein, the left marginal vein, the left posterior ventricular vein, the middle cardiac vein, and the small cardiac vein.

In the example of FIG. 1, the coronary sinus lead 106 includes a series of electrodes 123. In particular, a series of four electrodes are shown positioned in an anterior vein of the heart 102. Other coronary sinus leads may include a different number of electrodes than the lead 106. As described herein, an exemplary method selects one or more electrodes (e.g., from electrodes 123 of the lead 106) and determines characteristics associated with conduction and/or timing in the heart to aid in ventricular pacing therapy and/or assessment of cardiac condition. As described in more detail below, an illustrative method acquires information using various electrode configurations where an electrode configuration typically includes at least one electrode of a coronary sinus lead or other type of left ventricular lead. Such information may be used to determine a suitable electrode configuration for the lead 106 (e.g., selection of one or more electrodes 123 of the lead 106).

An exemplary coronary sinus lead 106 can be designed to receive ventricular cardiac signals (and optionally atrial signals) and to deliver left ventricular pacing therapy using, for example, at least one of the electrodes 123 and/or the tip electrode 122. The lead 106 optionally allows for left atrial pacing therapy, for example, using at least the left atrial ring electrode 124. The lead 106 optionally allows for shocking therapy, for example, using at least the left atrial coil electrode 126. For a complete description of a coronary sinus lead, the reader is directed to U.S. Pat. No. 5,466,254, “Coronary Sinus Lead with Atrial Sensing Capability” (Helland), which is incorporated herein by reference.

The stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the SVC coil electrode 134 will be positioned in the superior vena cava. Accordingly, the right ventricular lead 108 is capable of sensing or receiving cardiac signals, and delivering stimulation in the form of pacing and shock therapy to the right ventricle. An exemplary right ventricular lead may also include at least one electrode capable of stimulating other tissue; such an electrode may be positioned on the lead or a bifurcation or leg of the lead. A right ventricular lead may include a series of electrodes, such as the series 123 of the left ventricular lead 106.

FIG. 1 also shows a lead 160 as including several electrode arrays 163. In the example of FIG. 1, each electrode array 163 of the lead 160 includes a series of electrodes 162 with an associated circuit 168. Conductors 164 provide an electrical supply and return for the circuit 168. The circuit 168 includes control logic sufficient to electrically connect the conductors 164 to one or more of the electrodes of the series 162. In the example of FIG. 1, the lead 160 includes a lumen 166 suitable for receipt of a guidewire to facilitate placement of the lead 160. As described herein, any of the leads 104, 106, 108 or 110 may include one or more electrode arrays, optionally configured as the electrode array 163 of the lead 160. For example, the lead 106 may include features of the lead 160 and be suitable for multisite pacing for cardiac resynchronization therapy (CRT).

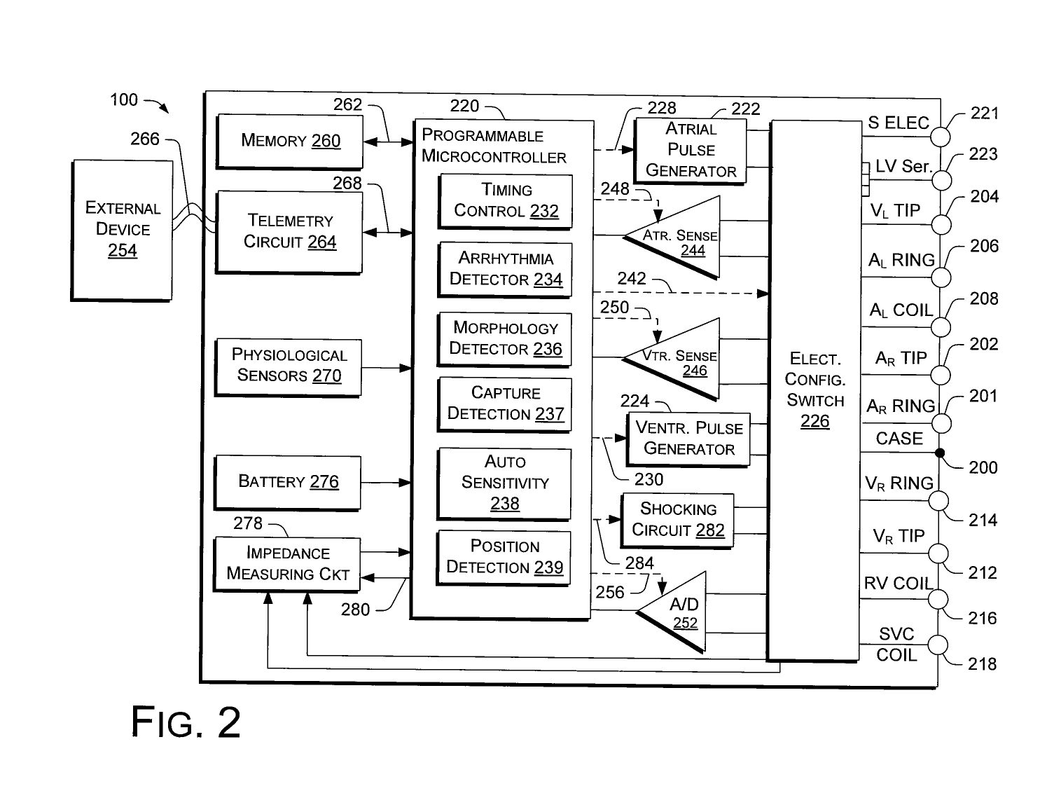

FIG. 2 shows an exemplary, simplified block diagram depicting various components of stimulation device 100. The stimulation device 100 can be capable of treating both fast and slow arrhythmias with stimulation therapy, including cardioversion, defibrillation, and pacing stimulation. While a particular multi-chamber device is shown, it is to be appreciated and understood that this is done for illustration purposes only. Thus, the techniques, methods, etc., described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart.

Housing 200 for the stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 for shocking or other purposes. Housing 200 further includes a connector (not shown) having a plurality of terminals 201, 202, 204, 206, 208, 212, 214, 216, 218, 221, 223 (shown schematically and, for convenience, the names of the electrodes to which they are connected are shown next to the terminals).

To achieve right atrial sensing, pacing and/or other stimulation, the connector includes at least a right atrial tip terminal (A_{R} TIP) 202 adapted for connection to the atrial tip electrode 120. A right atrial ring terminal (A_{R} RING) 201 is also shown, which is adapted for connection to the atrial ring electrode 121. To achieve left chamber sensing, pacing, shocking, and/or autonomic stimulation, the connector includes at least a left ventricular tip terminal (V_{L} TIP) 204, a left atrial ring terminal (A_{L} RING) 206, and a left atrial shocking terminal (A_{L} COIL) 208, which are adapted for connection to the left ventricular tip electrode 122, the left atrial ring electrode 124, and the left atrial coil electrode 126, respectively. Connection to suitable stimulation electrodes is also possible via these and/or other terminals (e.g., via a stimulation terminal S ELEC 221). The terminal S ELEC 221 may optionally be used for sensing. For example, electrodes of the lead 110 may connect to the device 100 at the terminal 221 or optionally at one or more other terminals.

A terminal 223 allows for connection of a series of left ventricular electrodes. For example, the series of four electrodes 123 of the lead 106 may connect to the device 100 via the terminal 223. The terminal 223 and an electrode configuration switch 226 allow for selection of one or more of the series of electrodes and hence electrode configuration. In the example of FIG. 2, the terminal 223 includes four branches to the switch 226 where each branch corresponds to one of the four electrodes 123.

To support right chamber sensing, pacing, shocking, and/or autonomic nerve stimulation, the connector further includes a right ventricular tip terminal (V_{R} TIP) 212, a right ventricular ring terminal (V_{R} RING) 214, a right ventricular shocking terminal (RV COIL) 216, and a superior vena cava shocking terminal (SVC COIL) 218, which are adapted for connection to the right ventricular tip electrode 128, right ventricular ring electrode 130, the RV coil electrode 132, and the SVC coil electrode 134, respectively.

At the core of the stimulation device 100 is a programmable microcontroller 220 that controls the various modes of cardiac or other therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that is suitable to carry out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052, the state-machine of U.S. Pat. Nos. 4,712,555 and 4,944,298, all of which are incorporated by reference herein. For a more detailed description of the various timing intervals used within the stimulation device and their inter-relationship, see U.S. Pat. No. 4,788,980, also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to autonomic nerves) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 232 to control the timing of the stimulation pulses (e.g., pacing rate, atrio-ventricular (AV) delay, interatrial conduction (AA) delay, or interventricular conduction (VV) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234. The detector 234 can be utilized by the stimulation device 100 for determining desirable times to administer various therapies. The detector 234 may be implemented in hardware as part of the microcontroller 220, or as software/firmware instructions programmed into the device and executed on the microcontroller 220 during certain modes of operation.

Microcontroller 220 further includes a morphology discrimination module 236, a capture detection module 237 and an auto sensing module 238. These modules are optionally used to implement various exemplary recognition algorithms and/or methods presented below. The aforementioned components may be implemented in hardware as part of the microcontroller 220, or as software/firmware instructions programmed into the device and executed on the microcontroller 220 during certain modes of operation. The capture detection module 237, as described herein, may aid in acquisition, analysis, etc., of information relating to IEGMs and, in particular, act to distinguish capture versus non-capture versus fusion.

The microcontroller 220 further includes an optional position detection module 239. The module 239 may be used for purposes of acquiring position information, for example, in conjunction with a device (internal or external) that may use body surface patches or other electrodes (internal or external). The microcontroller 220 may initiate one or more algorithms of the module 239 in response to a signal detected by various circuitry or information received via the telemetry circuit 264. Instructions of the module 239 may cause the device 100 to measure potentials using one or more electrode configurations where the potentials correspond to a potential field generated by current delivered to the body using, for example, surface patch electrodes. Such a module may help monitor positions of electrodes and/or cardiac mechanics in relationship to cardiac electrical activity and, in turn, may help to optimize cardiac resynchronization therapy based at least in part on such monitoring. The module 239 may operate in conjunction with various other modules and/or circuits of the device 100 (e.g., the impedance measuring circuit 278, the switch 226, the A/D 252, etc.).

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial and ventricular sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, the device 100 may utilize the atrial and ventricular sensing circuits, 244 and 246, to sense cardiac signals to determine whether a rhythm is physiologic or pathologic. Of course, other sensing circuits may be available depending on need and/or desire. In reference to arrhythmias, as used herein, “sensing” is reserved for the noting of an electrical signal or obtaining data (information), and “detection” is the processing (analysis) of these sensed signals and noting the presence of an arrhythmia or of a precursor or other factor that may indicate a risk of or likelihood of an imminent onset of an arrhythmia.

The exemplary detector module 234, optionally uses timing intervals between sensed events (e.g., P-waves, R-waves, and depolarization signals associated with fibrillation which are sometimes referred to as “F-waves”) and to perform one or more comparisons to a predefined rate zone limit (i.e., bradycardia, normal, low rate VT, high rate VT, and fibrillation rate zones) and/or various other characteristics (e.g., sudden onset, stability, physiologic sensors, and morphology, etc.) in order to determine the type of remedial therapy (e.g., anti-arrhythmia, etc.) that is desired or needed (e.g., bradycardia pacing, anti-tachycardia pacing, cardioversion shocks or defibrillation shocks, collectively referred to as “tiered therapy”). Similar rules can be applied to the atrial channel to determine if there is an atrial tachyarrhythmia or atrial fibrillation with appropriate classification and intervention.

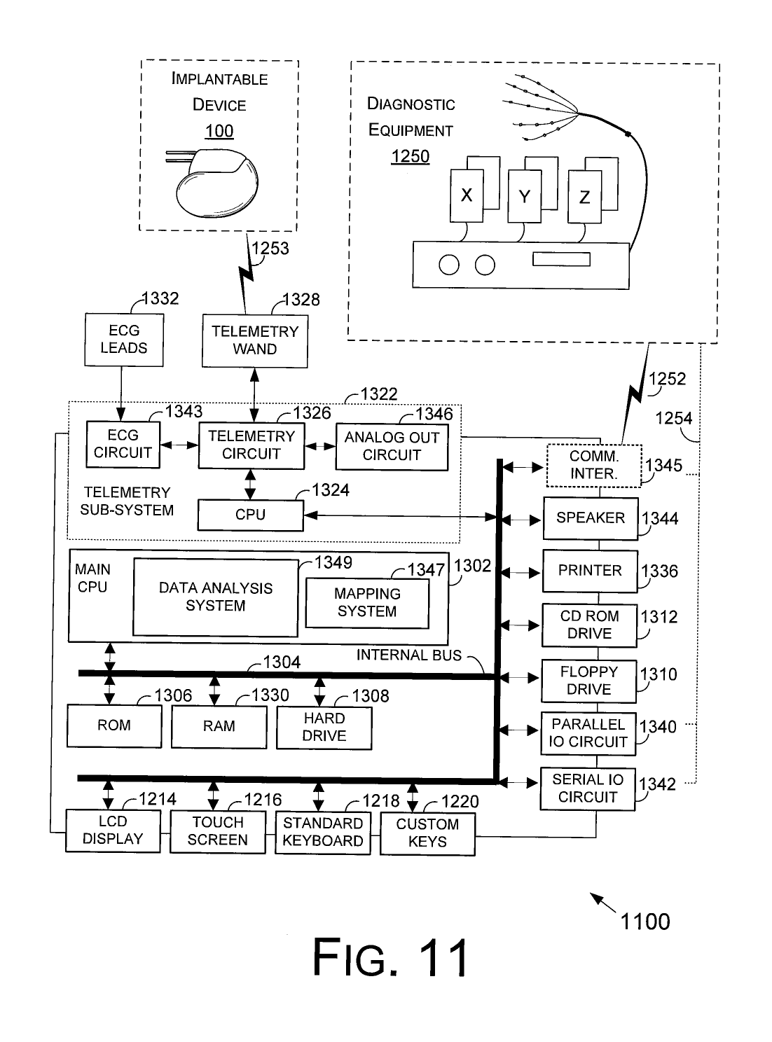

Cardiac signals are also applied to inputs of an analog-to-digital (ND) data acquisition system 252. Additional configurations are shown in FIG. 11 and described further below. The data acquisition system 252 is configured to acquire intracardiac electrogram (IEGM) signals or other action potential signals, convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or the nerve stimulation lead through the switch 226 to sample cardiac signals across any pair of desired electrodes. A control signal 256 from the microcontroller 220 may instruct the ND 252 to operate in a particular mode (e.g., resolution, amplification, etc.).

Various exemplary mechanisms for signal acquisition are described herein that optionally include use of one or more analog-to-digital converter. Various exemplary mechanisms allow for adjustment of one or more parameter associated with signal acquisition.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. One feature of the described embodiments is the ability to sense and store a relatively large amount of data (e.g., from the data acquisition system 252), which data may then be used for subsequent analysis to guide the programming of the device.

Advantageously, the operating parameters of the implantable device 100 may be non-invasively programmed into the memory 260 through a telemetry circuit 264 in telemetric communication via communication link 266 with the external device 254, such as a programmer, transtelephonic transceiver, or a diagnostic system analyzer. The microcontroller 220 activates the telemetry circuit 264 with a control signal 268. The telemetry circuit 264 advantageously allows intracardiac electrograms (IEGM) and other information (e.g., status information relating to the operation of the device 100, etc., as contained in the microcontroller 220 or memory 260) to be sent to the external device 254 through an established communication link 266.

The stimulation device 100 can further include one or more physiologic sensors 270. For example, the device 100 may include a “rate-responsive” sensor that may provide, for example, information to aid in adjustment of pacing stimulation rate according to the exercise state of the patient. However, the one or more physiological sensors 270 may further be used to detect changes in cardiac output (see, e.g., U.S. Pat. No. 6,314,323, entitled “Heart stimulator determining cardiac output, by measuring the systolic pressure, for controlling the stimulation,” to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), changes in the physiological condition of the heart, or diurnal changes in activity (e.g., detecting sleep and wake states). Accordingly, the microcontroller 220 responds by adjusting the various pacing parameters (such as rate, AV Delay, V-V Delay, etc.) at which the atrial and ventricular pulse generators, 222 and 224, generate stimulation pulses.

While shown as being included within the stimulation device 100, it is to be understood that one or more of the physiologic sensors 270 may also be external to the stimulation device 100, yet still be implanted within or carried by the patient. Examples of physiologic sensors that may be implemented in device 100 include known sensors that, for example, sense respiration rate, oxygen concentration of blood, pH of blood, CO₂ concentration of blood, ventricular gradient, cardiac output, preload, afterload, contractility, and so forth. Another sensor that may be used is one that detects activity variance, wherein an activity sensor is monitored diurnally to detect the low variance in the measurement corresponding to the sleep state. For a complete description of the activity variance sensor, the reader is directed to U.S. Pat. No. 5,476,483 which is hereby incorporated by reference.

The one or more physiological sensors 270 optionally include sensors for detecting movement and minute ventilation in the patient. Signals generated by a position sensor, a MV sensor, etc., may be passed to the microcontroller 220 for analysis in determining whether to adjust the pacing rate, etc. The microcontroller 220 may monitor the signals for indications of the patient's position and activity status, such as whether the patient is climbing upstairs or descending downstairs or whether the patient is sitting up after lying down.

The stimulation device 100 additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 200 V, for periods of 10 seconds or more). The battery 276 also desirably has a predictable discharge characteristic so that elective replacement time can be detected.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. The impedance measuring circuit 278 is advantageously coupled to the switch 226 so that any desired electrode may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to 0.5 J), moderate (e.g., 0.5 J to 10 J), or high energy (e.g., 11 J to 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode).

Cardioversion level shocks are generally considered to be of low to moderate energy level (so as to minimize pain felt by the patient), and/or synchronized with an R-wave and/or pertaining to the treatment of tachycardia. Defibrillation shocks are generally of moderate to high energy level (e.g., corresponding to thresholds in the range of approximately 5 J to 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

As already mentioned, the implantable device 100 includes impedance measurement circuitry 278. Such a circuit may measure impedance or electrical resistance through use of various techniques. For example, the device 100 may deliver a low voltage (e.g., about 10 mV to about 20 mV) of alternating current between the RV tip electrode 128 and the case electrode 200. During delivery of this energy, the device 100 may measure resistance between these two electrodes where the resistance depends on any of a variety of factors. For example, the resistance may vary inversely with respect to volume of blood along the path.

In another example, resistance measurement occurs through use of a four terminal or electrode technique. For example, the exemplary device 100 may deliver an alternating current between one of the RV tip electrode 128 and the case electrode 200. During delivery, the device 100 may measure a potential between the RA ring electrode 121 and the RV ring electrode 130 where the potential is proportional to the resistance between the selected potential measurement electrodes.

With respect to two terminal or electrode techniques, where two electrodes are used to introduce current and the same two electrodes are used to measure potential, parasitic electrode-electrolyte impedances can introduce noise, especially at low current frequencies; thus, a greater number of terminals or electrodes may be used. For example, aforementioned four electrode techniques, where one electrode pair introduces current and another electrode pair measures potential, can cancel noise due to electrode-electrolyte interface impedance. Alternatively, where suitable or desirable, a two terminal or electrode technique may use larger electrode areas (e.g., even exceeding about 1 cm²) and/or higher current frequencies (e.g., above about 10 kHz) to reduce noise.

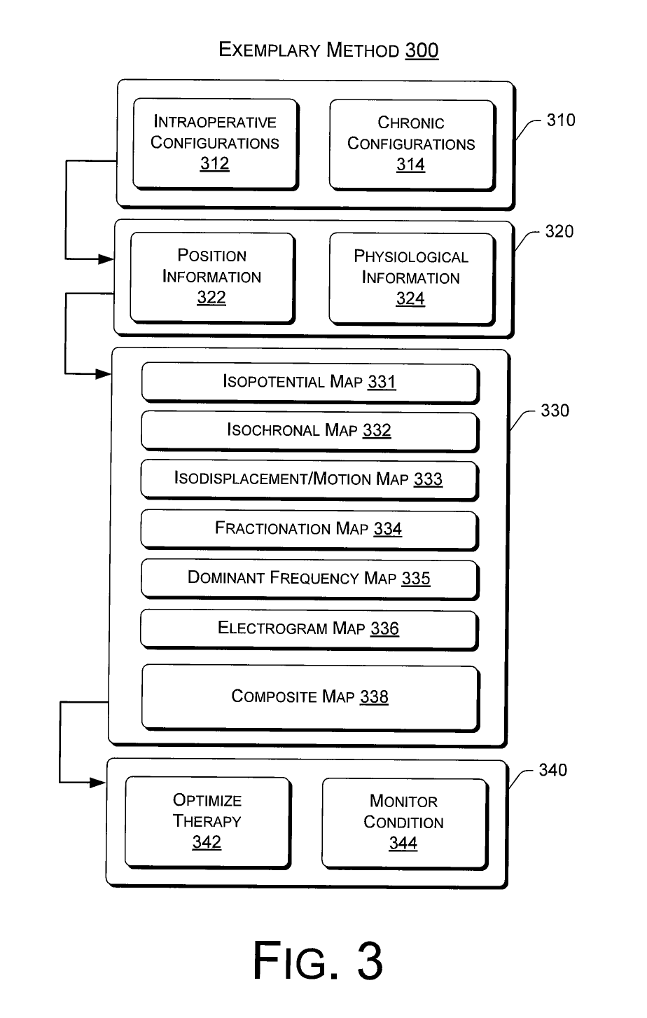

FIG. 3 shows an exemplary method 300 for acquiring information and generating one or more maps. In the example of FIG. 3, the method 300 includes a configurations block 310 that includes intraoperative configurations 312 and chronic configurations 314. The intraoperative configurations 312 pertain to configurations that may be achieved during an operative procedure. For example, during an operative procedure, one or more leads may be positioned in a patient where the one or more leads are connected to, or variously connectable to, a device configured to acquire information and optionally to deliver energy to the patient (e.g., to the heart, to a nerve, to other tissue, etc.). Such energy may be for purposes of blocking conduction or activation, stimulation, shock or ablation. The chronic configurations 314 pertain to configurations achievable by a chronically implanted device and, for example, one or more associated leads. In general, intraoperative configurations include those achievable by physically re-positioning a lead in a patient's body while chronic configurations normally do not allow for re-positioning as a lead or leads are usually anchored during implantation or become anchored in the weeks to months after implantation. Chronic configurations do, however, include selection of a subset of the multiple implanted electrodes, for example using a tip electrode versus a particular ring electrode as a cathode or using the tip electrode and a ring electrode as a bipolar pair versus using the tip electrode and the ring electrode as two independent cathodes. Thus, intraoperative configurations include configurations available by changing device settings, electrode selection, and physical position of electrodes, while chronic configurations include only those configurations available by changing device settings and electrode selection, or “electronic repositioning” of one or more stimulation electrodes.

As indicated in FIG. 3, an acquisition block 320 includes acquisition of position information 322 and acquisition of physiological information 324 (e.g., electrical information as to electrical activity of the heart, biosensor information, etc.). While an arrow indicates that a relationship or relationships may exist between the configurations block 310 and the acquisition block 320, acquisition of information may occur by using in part an electrode (or other equipment) that is not part of a configuration. For example, the acquisition block 320 may rely on one or more surface electrodes that define a coordinate system or location system for locating an electrode that defines one or more configurations. For example, three pairs of surface electrodes positioned on a patient may be configured to deliver current and define a three-dimensional space whereby measurement of a potential locates an electrode in the three-dimensional space.

As described herein, an electrode may be configured for delivery of energy to the heart; for acquisition of electrical information; for acquisition of position information; for acquisition of electrical information and position information; for delivery of energy to the heart and for acquisition of electrical information; for delivery of energy to the heart and for acquisition of position information; for delivery of energy to the heart, for acquisition of electrical information and for acquisition of position information.

In various examples, acquisition of position information occurs by measuring one or more potentials where the measuring relies on an electrode that may also be configured to deliver energy to the heart (e.g., electrical energy to pace a chamber of the heart). In such a scenario, the electrode may deliver energy sufficient to stimulate the heart and then be tracked along one or more dimensions to monitor the mechanical consequences of the stimulation. Further, such an electrode may be used to acquire electrical information (e.g., an IEGM that evidences an evoked response). Such an electrode can perform all three of these tasks with proper circuitry and control. For example, after delivery of the energy, the electrode may be configured for acquiring one or more potentials related to location and for acquiring an electrogram. To acquire potentials and an electrogram, circuitry may include gating or other sampling techniques (e.g., to avoid circuitry or interference issues). Such circuitry may rely on one sampling frequency for acquiring potentials for motion tracking and another sampling frequency for acquiring an electrogram.

The method 300 of FIG. 3 includes a determination block 330 for determining one or more maps. Some exemplary maps are presented in block 330. Specifically, block 330 includes an isopotential map 331, an isochronal map 332, an isodisplacement map or isomotion map 333, a fractionation map 334, a dominant frequency map 335, an electrogram map 336 and a composite map 338, which may be based one or more of the maps 331-336 or another type of map or maps.

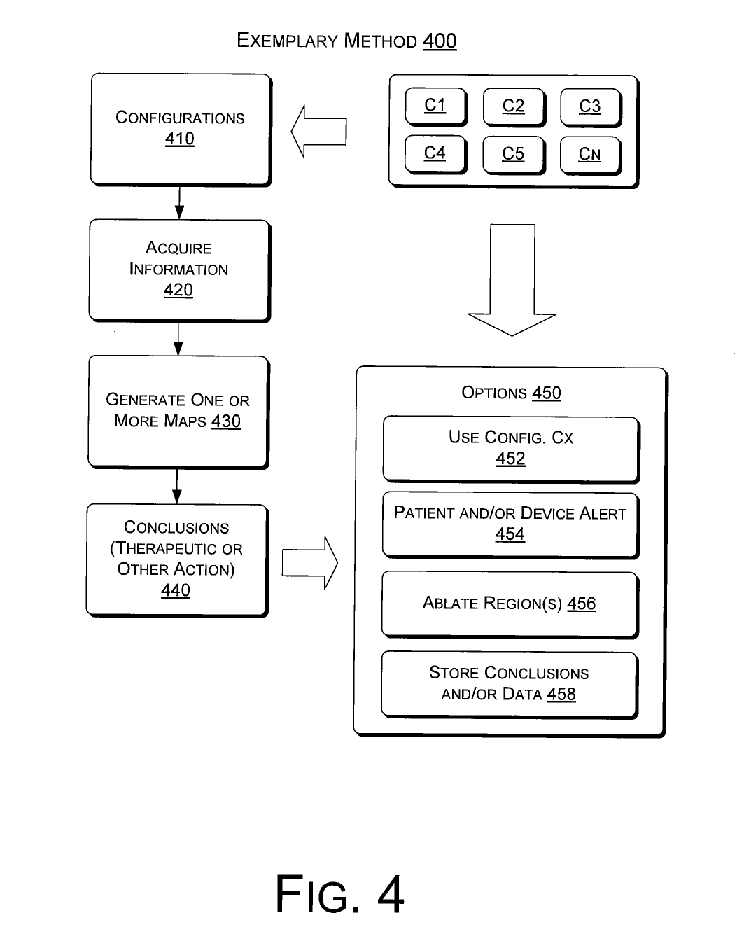

As shown in the example of FIG. 3, the conclusion block 340 may perform actions such as to optimize therapy 342 (e.g., ablation, CRT, etc.) and/or to monitor patient and/or device condition 344. These options are described in more detail with respect to FIG. 4. Before describing FIG. 4, some details are provided for the maps of block 330.

The ENSITE® localization system is configured to acquire electrical potentials associated with cardiac activity and generate and display a color-coded isopotential map. For example, with the ENSITE® multielectrode array catheter, the ENSITE® system can acquire electrical potentials associated with right atrial activity, generate a map and display the map to thereby allow a clinician to identify a region that may be a source of an arrhythmia. In general, a region with low potential (e.g., <0.5 mV) may be relatively inactive or scar tissue, noting that various types of arrhythmias occur in association with inactive or scar tissue. In another example, with the ENSITE® NAVX® surface electrodes, the ENSITE® system can locate and display positions of conventional electrophysiology catheters in the body, particularly, in or around the heart.

The isopotential map 331 may rely on potential, peak-to-peak potential, negative peak, positive peak or other potentials. For example, an isopotential map may show local peak-to-peak IEGM amplitude where a scar is depicted by low potential (i.e., voltage) isopotentials or contoured region. A surface area calculation may determine a surface area or volume of the scar region, which can be optionally compared to a total myocardial surface area or volume of a chamber of the heart.

The isochronal or isochrone map 332 may be based on activation times for different regions of the heart. For example, an isochronal map may be generated based on times for QRS complex on-set as noted in multi-site IEGM data. In such a map, isochrones may be mapped at regular intervals (e.g., 5 ms to 25 ms), which may account for characteristics of arrhythmic behavior.

Monomorphic ventricular tachycardia (VT) often results from either an increased automaticity of a single ventricular site or a reentrant circuit. A common cause of monomorphic VT is damaged or scar tissue (e.g., from a previous myocardial infarction). As a scar exhibits abnormal electrical response, a potential circuit often exists around the scar that can cause tachycardia. Rarer congenital causes of monomorphic VT include right ventricular dysplasia and right and left ventricular outflow tract VT.

For normal sinus activity at 60 beats per minute and isochrones that span 0 ms to 1,000 ms (0 ms representing P-wave onset), a ventricular scar region will typically be surrounded by isochrones well beyond the time of a normal P-R interval (e.g., >200 ms).

Another type of ischronal map shows electrical conduction velocity for regions of the heart. For example, where the distance between two electrodes is known, the distance may be used in conjunction with IEGM event times to determine a local conduction velocity.

The isodisplacement map or isomotion map 333 is based on localized movement of the myocardium. With respect to displacement, the ENSITE® NAVX® system may measure displacement of an electrode during a cardiac cycle and determine a maximum displacement or a displacement for a particular portion of the cardiac cycle. Displacement may be optionally specified with respect to a direction of a Cartesian coordinate system (e.g., x, y, z), a cylindrical coordinate system (e.g., r, z, Θ) or other coordinate system (e.g., along major and minor axis of a ventricle). Alternatively, direction may be coded and a map generated based on direction (e.g., a different color for each direction).

Motion refers to movement with respect to time and can include velocity (i.e., isovelocity) and acceleration (i.e., isoacceleration). Thus, an isomotion map may show velocity contours based, for example, on maximum velocity to indicate which regions move faster than others. Velocity may be further categorized as diastolic velocity or systolic velocity. Hence, an isomotion map may be generated and displayed for maximum diastolic velocity and an isomotion map may be generated and displayed for maximum systolic velocity. Such distinctions may be made for displacement as well.

The fractionation map 334 shows fractionation of electrical activity of the heart. Fractionation may be defined as an electrical activity signal (e.g., electrogram) having a certain number of deflections over a specified time period, over a cardiac cycle or over a portion of a cardiac cycle. Fractionation may be structural, functional or a combination thereof. For example, functional fractionation may be evidence of multiple wavefronts in a region due to multiple sites of activation, which may be natural, artificial or a combination of natural and artificial. In contrast, structural fractionation arises from tissue abnormalities and often indicates presence of damaged myocardium or scarring (e.g., also consider that a local fractionated IEGM may have low amplitude). In some instances, increased fractionation can be a precursor of VT. Regions with fractionated IEGMs may indicate viable muscle fibers and strands of fibrous tissue are interwoven or where muscle fibers are isolated by strands of connective tissue. Fractionation may increase with the number of isolated fiber bundles near an IEGM acquisition site. A fractionation map may also help characterize VT as being monomorphic or polymorphic.

The ENSITE® system includes fractionation mapping features (complex fractionated electrograms mapping tool) that can assist in diagnosis of arrhythmias such as atrial fibrillation. The complex fractionated electrograms (CFE) mapping tool can objectively detects areas of complex fractionated electrograms and display color-coded findings with respect to a chamber model. The CFE mapping tool can categorize higher and lower mean value electrograms and standard deviations (e.g., indicative of interval regularity) and map such metrics with respect to diagnostic landmarks. The ENSITE® system can also provide for acquisition from multiple electrodes simultaneously.

As described herein, the fractionation map 334 may include electrical signal fractionation information, mechanical signal fractionation information or a combination of electrical signal fractionation information and mechanical signal fractionation information. For example, an isofractionation map for motion may be generated based on analysis of motion waveforms for one or more regions of the heart. An exemplary method may generate a composite map that maps electrical and mechanical fractionation. Such a composite map may aid in understanding underlying causes of fractionation. Where various electrical stimulation schemes (e.g., various electrode positions, configurations, stimulation parameters, etc.) are implemented, such a composite map or maps may allow a clinician to understand better underlying causes of fractionated electrical or mechanical signals and optionally aid the clinician in placement of electrodes, stimulation settings, etc., to optimize cardiac performance.

The dominant frequency (DF) map 335 shows local dominant frequencies. As for fractionation analyses, dominant frequency analyses may be applied to electrical waveforms, motion waveforms or a combination of both. A high dominant electrical frequency (e.g., a single frequency peak) can be indicative of a rotor while multiple frequency peaks over a wide range of frequencies can be indicative of abnormal conduction (e.g., a conduction block). An abnormal DF map may be due to structural, functional or a combination of structural and functional causes. Abnormal regions on a DF map may be associated with occurrence and maintenance of arrhythmias (see, e.g., Umapathy, “Anatomic Substrate as Determinant of Dominant Frequency Dynamics During Human Ventricular Fibrillation,” Circulation. 2008; 118:S_{—}927-S_{—}928: “50% of max-min DF frequencies locations match the scar locations and in 97% of the matched locations the max-min DF occur at the vicinity of the scar”).

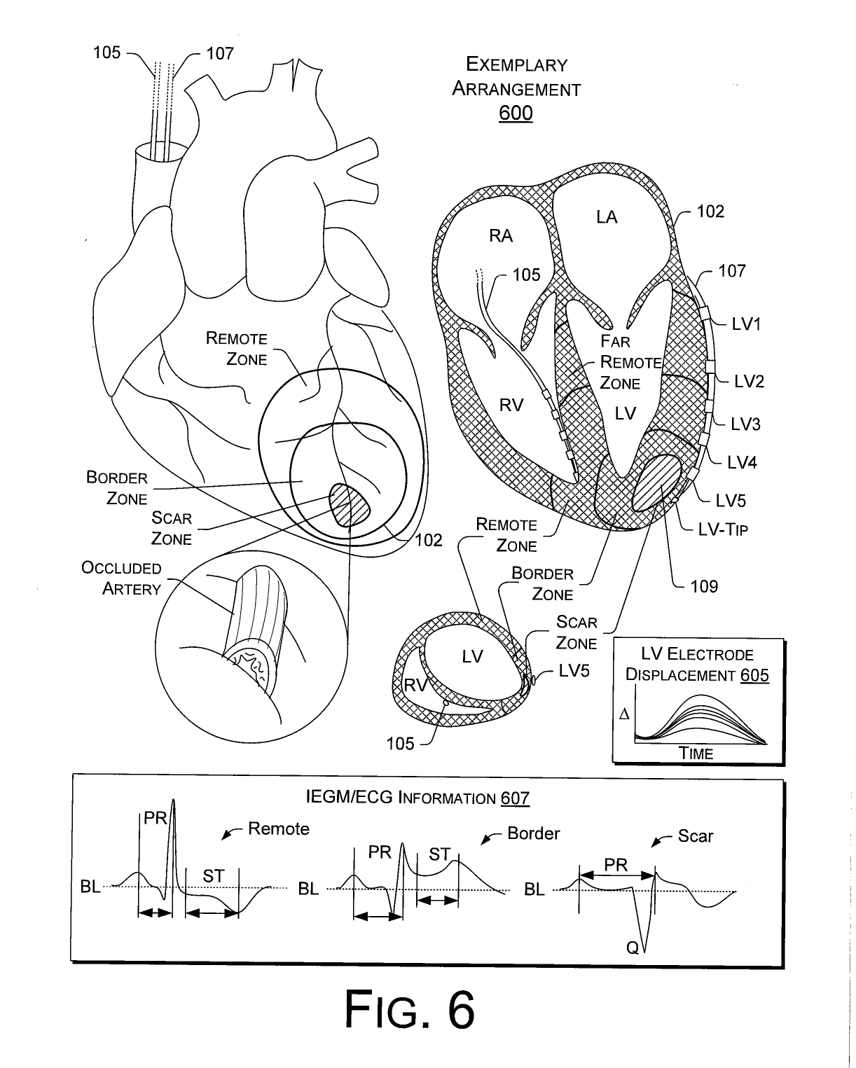

The electrogram map 336 may include any of a variety of electrogram information. For example, such a map may include ST segment elevation, ST segment depression, PR segment prolongation, PR segment depression, T wave morphology or Q wave morphology. Various electrogram analyses are shown with respect to FIG. 6, for example, where electrogram information can be indicative of cardiac health.

As described herein, the composite map 338 shows two or more different metrics overlain and optionally weighted to identify damaged myocardium or scars. Various examples are discussed below. An individual map or a composite map may be animated to demonstrate electrical, mechanical or a combination of electrical and mechanical information that occurs with respect to time. For example, electrical activity around a scar may be animated and resemble a marquee. Similarly, mechanical motion around a scar may be animated. In combination, electrical and mechanical information can facilitate identification of damaged myocardial regions and scars.

A composite map may be based on data acquired with respect to one or more morphologies (e.g., arrhythmia, P-wave, QRS complex, etc.), a portion of a cardiac cycle (e.g., systolic, diastolic), or one or more cardiac cycles. A composite map may include one type of data acquired with respect to morphology and other type of data acquired with respect to a portion of a cardiac cycle. For example, electrical activation data for a QRS complex may be used to determine peak amplitude of the QRS complex while displacement data for an entire cardiac cycle is used to determine maximum displacement.

With respect to weighting of a metric, an exemplary method may weight one or more of an isopotential map, an isochronal map and a motion map. For example, an isopotential map and an isochronal map may be weighted to contribute less to a composite map that includes isopotential, ischronal, and isomotion data (i.e., to attribute more weight to motion). Specifically, electrical activity is function of substrate, which, at a given point in time, may have built-up characteristics (e.g., due to reentrant activation). Such built-up characteristics may be considered a functional “obstacle” and not an anatomical obstacle, which, in turn, introduces some uncertainty to scar identification based solely on electrical activity data.

As scars are anatomical obstacles that cannot move on their own, motion of healthy tissue is unlikely to mimic characteristics of a scar (e.g., which may simply move due to tethering). A scar may also move in a direction that is not aligned with surrounding tissue (e.g., consider a bulge that may move in an opposite direction). Further, as a scar typically has a lower potential (e.g., peak-to-peak IEGM) and exhibits less motion than healthy tissue, a composite map generated from isopotential data and isomotion data can assist a clinician in identifying scars. Further, a system may include a module that relies on one or more criteria (e.g., potential and motion criteria) to automatically highlight or otherwise identify regions as potentially being damaged or scarred. With respect to automatic identification, an appropriate selection of criteria may replace a weighting scheme that weights electrical data different from mechanical data (e.g., to rely more heavily on the mechanical data).

As described herein, an ENSITE® NAVX® mapping technique can help characterize myocardial viability. For example, ENSITE® system motion mapping can be performed in conjunction with traditional mapping techniques (e.g., electroanatomical mapping such as voltage and activation sequence mapping) to enhance mapping capability for detection of damaged myocardium or scars during an EP procedure or during an implant procedure. Where suitably configured, a system may be capable of performing such mapping using a localization system and a chronically implanted device or a suitably configured chronically implanted device. Various exemplary methods described herein may be used for purposes of ablation, chronic device implantation, therapy optimization, etc.

As mentioned, the ENSITE® NAVX® system can determine the position of electrodes within the cardiac space. By tracking electrode positions for different areas of the heart (either simultaneously or sequentially), a motion map can be generated that can identify or assist in identification of area(s) with small or no motion, indicative of scar areas.

As described herein, catheter mapping techniques of the ENSITE® NAVX® system can be used to map voltage and activation sequence as isopotentials and isochrones. With respect to isopotential data, bipolar voltage amplitudes less than about 1.5 mV may be identified as corresponding to low voltage zones while bipolar voltage amplitudes less than about 0.5 mV may be identified as corresponding to scar zones. A mapping module may render these zones to a display along with a map that identifies anatomical features of the heart.

With respect to isochronal data, myocardial areas with slow conduction and conduction block may be identified due to crowding of isochrones. Areas with potential substrate abnormalities (e.g., scarring) may be identified also by analyzing wave front propagation. Further, reentrant like activation may be noted and mapped. Specifically, an exemplary module may display a user interface where degree of reentrant circuit (e.g., wave front curvature, head and tail interaction, wavelength) can be user selectable to identify such areas.

Voltage and activation sequence mapping can also be accomplished epicardially via a transvenous approach (with some limitations) and/or via an intrapericardial approach (to provide mapping with fewer limitations).

A motion map with endocardial and/or epicardial contours may be generated using an EP catheter(s) and/or lead(s). Criteria may be user selected or preprogrammed to identify areas with no, low, moderate, and high motion. A mapping module may render these areas as a contour map in conjunction with anatomical features of the heart where areas with no and low motion can be marked as possible scar areas on the contour map. An exemplary map may include displacement and/or motion. For example, extent of motion can be expressed as displacement in absolute magnitude, as in a peak-to-peak distance of excursion, or as a percent of magnitude relative to the greatest magnitude measured in the heart or in a region of the heart. Further, extent of motion or displacement in a specific direction, plane, quadrant, etc., may be of interest to a clinician. An exemplary module can render a user interface to a display that allows a user to select directions or other parameters with respect to motion or displacement. An exemplary technique may determine (e.g., in addition to overall displacement magnitude) extent of radial motion or the extent of twist (e.g., using a coil or spring model of a ventricle), which may be a more sensitive or specific indicator of a scar region than the overall magnitude alone.

With respect to extent of motion, an exemplary module may categorize motion data as corresponding to categories for no motion, low extent of motion, moderate extent of motion and high extent of motion. A user interface may allow a user to select criteria for making such categorizations, optionally on a chamber-by-chamber basis (e.g., to more finely illustrate motion of the right atrium compared to the left ventricle).

An exemplary module may map direction of motion relative to adjacent tissue to help delineate scar from healthy myocardium. For example, during systole normal myocardium will display inward radial motion, while scar may demonstrate outward radial motion or “bulging.” The exemplary module may identify areas that have such dyskinesis, outline these areas and identify them as being scar tissue. An exemplary module may analyze motion data to determine the timing of motion for a region of myocardium relative to motion of one or more neighboring regions. As described herein, correlation between direction of motion (or extent of motion) and motion timing can help to identify scar tissue that may have moderate or high motion due to passive tethering rather than due to its own active contraction (e.g., consider a time lag due to mechanics of tethering).

An exemplary module may analyze electrical activation data to determine the timing of electrical activation for a region of myocardium relative to timing of electrical activation of one or more neighboring regions. As described herein, electrical activation data and displacement or motion data may be analyzed for correlations that allow for differentiation of damaged or scarred tissue and healthier tissue. For example, where scar tissue moves due to tethering, activation of neighboring healthier tissue should occur just prior to motion of scar tissue due to tethering. In particular, areas of low voltage and later activation, whose motion is moderate and early, would be identified as scar. Areas whose motion shows greater concordance with neighboring tissue electrical activation than with its own electrical activation may also be identified as scar.

As mentioned, composite maps can be generated based on various metrics and can assist clinicians in identifying damaged or scarred tissue. Such maps may be in three-dimensions in space and include a time as a fourth dimension. To generate a composite map of disparate data, the data should be registered to a common coordinate system. For example, if electrical activity data is acquired along with position information associated with a coordinate system of one type of localization system and motion data (e.g., position data with respect to time) is acquired as associated with a coordinate system of another type of localization system, the electrical activity data or the motion data should be registered to a common coordinate system (e.g., one or the other or a third coordinate system) prior to generation of a composite map (e.g., using fiducial markers or anatomical markers). Where a particular localization system is used (e.g., the ENSITE® NAVX® system), acquired data may be associated with a common coordinate system, which can facilitate generation of a composite map.

An exemplary method acquires data for two or more metrics that can indicate possible scarring of the myocardium and presents the data in a single map, which may be labeled as a “scar” map. Such a method may first generate individual maps for each of the metrics and then generate a composite map by overlaying the maps. The overlaid maps may form a composite map that is generated by drawing borders that delineate data of each map on an anatomic map. In such a composite map, a scar may be identified as a region representing a union of two or more borders. Alternatively, the overlaid maps may form a composite map that is generated by shading an anatomic surface with a color scale corresponding to relative values of corresponding map parameters (e.g., voltage, activation time, motion, DF, CFE, etc). An exemplary module optionally allow for assigning transparency values to each individual layer (e.g., metric) of a composite map. Such a module may render to a display a region where layers overlap, which may indicate scarring. For example, a delineated region may correspond to color values where a first layer color and a second layer color overlap (e.g., blue and yellow to form green) or where common colors overlap to decrease intensity (e.g., purple and purple to form dark purple).

In addition to generating overlaid maps of individual scar identification parameters, composite maps can be generated as representing a pre-determined mathematical aggregation of two or more metrics. For example, the ratio of potential amplitude (e.g., in mV, peak-to-peak for a QRS complex) to motion amplitude (e.g., in mm, over a cardiac cycle) can allow for identification of scar or hibernating regions whose motion is due to tethering effects.

FIG. 4 shows the exemplary method 400 with various configurations 410 (C1, C2, . . . , Cn) and options 450. As mentioned, a configuration may be defined based on factors such as electrode position (e.g., with respect to some physiological feature of the heart or another electrode), sensor position, stimulation parameters for an electrode or electrodes and, where appropriate, one or more interelectrode timings. A configuration may correspond to a condition such as instructing a patient to hold her breath, administration of an agent, tilt with respect to gravity, etc.

With reference to FIG. 1, C1 may be a configuration that relies on the RV tip electrode 128, the RV ring electrode 130, the LV tip electrode 122 and the LV ring electrode 124 while C2 may be a configuration that relies on the same electrodes as C1 but where the stimulation polarity for the LV electrodes is reversed. Further, C3 may rely on the same electrodes where the timing between delivery of a stimulus to the RV and delivery of a stimulus to the LV is different compared to C1. Yet further, C4 may rely on the same electrodes where the duration of a stimulus to the RV is different compared to C1. In these foregoing examples, configurations provide for one or more electrodes to deliver energy to stimulate the right ventricle and for one or more electrodes to deliver energy to stimulate the left ventricle. In other examples, configurations may provide for stimulation of a single chamber at one or more sites, stimulation of one chamber at a single site and another chamber at multiple sites, multiple chambers at multiple sites per chamber, etc.

In an acquisition block 420, acquisition occurs for physiological information and position information or solely position information (e.g., with respect to time) where such information pertains to one or more configurations. In a generation block 430, one or more maps are generated based on one or more measures or metrics, which are based at least in part on the acquire information. A conclusions block 440 provides for therapeutic or other action, which may be selected from one or more options 450.

In the example of FIG. 4, the one or more options 450 include selection of a configuration 452 (e.g., Cx, where x is a number selected from 1 to n), issuance of a patient and/or device alert 454 that pertains to condition of a patient or a condition of a device or associated lead(s) or electrode(s), issuance of an indication as to a region or regions to ablate or otherwise treat 456 and storage of conclusion(s) and/or data 458. The options 450 may be associated with the configurations 410, as indicated by an arrow. For example, storage of conclusions and/or data 456 may also store specific configurations, a generalization of the configurations (e.g., one or more shared characteristics), a device/system arrangement (e.g., where the number and types of configurations would be known based on the arrangement), etc.

As described herein, an exemplary method can include: positioning one or more electrodes within the heart and/or surrounding space (e.g., intra-chamber, intra-vascular, intrapericardial, etc., which may be collectively referred to as “cardiac space”); and acquiring position information (e.g., via one or more measured potentials) to determine a location, locations or displacement for at least one of the one or more electrodes using an electroanatomic mapping system (e.g., the ENSITE® NAVX® system or other system with appropriate features). Such a method may also include acquiring physiological information. For example, positioned electrodes or sensors may be configured for acquisition of electrical information (e.g., IEGMs) or other physiological information (e.g., pH, gas concentration, etc.). Further, with respect to acquisition of information, an acquisition system may operate at an appropriate sampling rate. For example, an acquisition system for position information may operate at a sampling rate of about 100 Hz (e.g., the ENSITE® NAVX® system can sample at about 93 Hz) and an acquisition system for electrical information may operate at a sampling rate of about 1200 Hz (e.g., in unipolar, bipolar or other polar arrangement). In general, a sensor will have an associated sampling rate, which may be less than or greater than the sampling rate for position information.

An exemplary method may include preparing a patient for both implant of a device such as the device 100 of FIGS. 1 and 2 or for treatment using a catheter (e.g., an ablation catheter) and for electroanatomic mapping study. Such preparation may occur in a relatively standard manner for the implant or treatment and for using the ENSITE® NAVX® system or other similar technology. As described herein, any of a variety of electroanatomic mapping or locating systems that can locate indwelling electrodes in and around the heart may be used.

Once prepped, a clinician or robot may place leads and/or catheters in the patient's body, including any leads to be chronically implanted as part of a CRT system, as well as optional additional electrodes that may yield additional information (e.g., to increase accuracy by providing global information or other information).

After an initial placement of an electrode-bearing catheter or an electrode-bearing lead, a clinician may then connect one or more electrodes to an electroanatomic mapping or locating system. The term “connection” can refer to physical electrical connection or wireless connection (e.g., telemetric, RF, ultrasound, etc.) with the electrodes or wireless connection with another device that is in electrical contact with the electrodes.

Once an appropriate connection or connections have been made, real-time position data for one or more electrodes may be acquired for various configurations or conditions. For example, position data may be acquired during normal sinus rhythm; pacing in one or more chambers; advancing, withdrawing, or moving a location of an electrode; pacing one or more different electrode configurations (e.g. multisite pacing); varying inter-stimulus timing (e.g. AV delay, VV delay); administering an agent; tilting a patient; etc.

In various examples, simultaneous to the position recording, an intracardiac electrogram (IEGM) from each electrode can also be recorded and associated with the anatomic position of the electrode. While various examples refer to simultaneous acquisition, acquisition of electrical information and acquisition of mechanical information may occur sequentially (e.g., alternate cardiac cycles) or interleaved (e.g., both acquired during the same cardiac cycle but offset by sampling time or sampling frequency).

In various exemplary methods, electrodes within the cardiac space may be optionally positioned at various locations (e.g., by continuous movement or by discrete, sequential moves), with a mapping system recording the real-time motion information at each electrode position in a point-by-point manner. Such motion data can by associated with a respective anatomic point from which it was collected. By moving the electrodes from point to point during an intervention, the motion data from each location can be incorporated into a single map, model, or parameter.

As explained, an exemplary method may include determining one or more metrics. In turn, an algorithm or a clinician may select a configuration (e.g., electrode location, multisite arrangement, AV/VV timing, ablation, etc.) based on the one or more determined metrics. A chronic configuration may be optionally updated from time to time (e.g., during a follow-up visit, in a patient environment, etc., depending on specific capabilities of a system).

An exemplary method may rely on certain equipment at time of implant or exploration and other equipment after implantation of a device to deliver a cardiac therapy. For example, during an intraoperative procedure, wireless communication may not be required; whereas, during a follow-up visit, measured potentials for position of chronically implanted electrodes (e.g., position information) and of measured IEGMs using chronically implanted electrodes (e.g., physiological information) may be communicated wirelessly from an implanted device to an external device. With respect to optimization of a chronically implanted system, in general, electrode location will not be altered, but other parameters altered to result in an optimal configuration (e.g., single- or multi-site arrangement, polarity, stimulation energy, timing parameters, etc.).

As discussed herein, various exemplary techniques deliver current and measure potential where potential varies typically with respect to cardiac mechanics (e.g., due to motion). For example, electrodes for delivery of current may be placed at locations that do not vary significantly with respect to cardiac mechanics while one or more electrodes for measuring potential may be placed at a location or locations that vary with respect to cardiac mechanics. Alternatively, electrodes for measuring potential may be placed at locations that do not vary significantly with respect to cardiac mechanics while one or more electrodes for delivery of current may be placed at a location or locations that vary with respect to cardiac mechanics. Various combinations of the foregoing arrangements are possible as well. Electrodes may be associated with a catheter or a lead. In some instances, an electrode may be a “stand-alone” electrode, such as a case electrode of an implantable device (see, e.g., the case electrode 200 of the device 100 of FIGS. 1 and 2).

In accordance with the method 300 of FIG. 3 and the method 400 of FIG. 4, an exemplary method may include preparing a patient for both implant and an electroanatomic mapping study. In this example, preparation can be accomplished in standard manner for implant preparation and the mapping may rely on a localization system such as the ENSITE® NAVX® system or other similar technology for the mapping prep. After preparing the patient, the method includes placing leads and/or catheters in the patient's body, including any leads to be chronically implanted as part of the CRT system, as well as optional additional electrodes that will yield more information, for example, to thereby increase versatility of mechanical dyssynchrony determinations. After placement, the method includes connecting electrodes on leads and/or catheters to the localization system (e.g., electroanatomic mapping system). With respect to the term “connecting”, depending on the equipment, it may include physical electrical connecting and/or telemetric/RF/wireless/ultrasound/other communication connecting (e.g., directly or indirectly, via another “bridging” device, with the electrodes.)

After appropriate connections are made, acquiring or recording follows to record real-time positions of one or more electrodes for various configurations or conditions such as, but not limited to: normal sinus rhythm; pacing in one or more chambers (e.g., RV pacing, LV pacing BiV pacing); at various lead placement locations, (i.e., advancing, withdrawing, or moving the location of an electrode); pacing one or more different electrode configurations (e.g. multisite pacing); or varying inter-stimulus timing (e.g. AV delay, W delay). After or during acquisition, the method can determine one or more metrics, which may be mapped in individual maps or in one or more composite maps. Subsequently, based on one or more of the metrics, optionally in conjunction with other information (e.g., other ENSITE® real-time cardiac performance parameters), a clinician or a device may select a configuration (e.g., electrode location, multisite configuration, AV/VV delays, etc.) that yielded or yields the best value(s) for cardiac performance. This configuration may then be used chronically (e.g., as the final configuration of the CRT setup).

Such a method may separately be implemented at a clinic or hospital follow-up after the time of implant, provided wireless communication with the chronic indwelling electrodes. In general, it can be assumed that the electrode location will not be altered, but optimization of single- or multi-site configuration as well as timing parameter may still be performed.

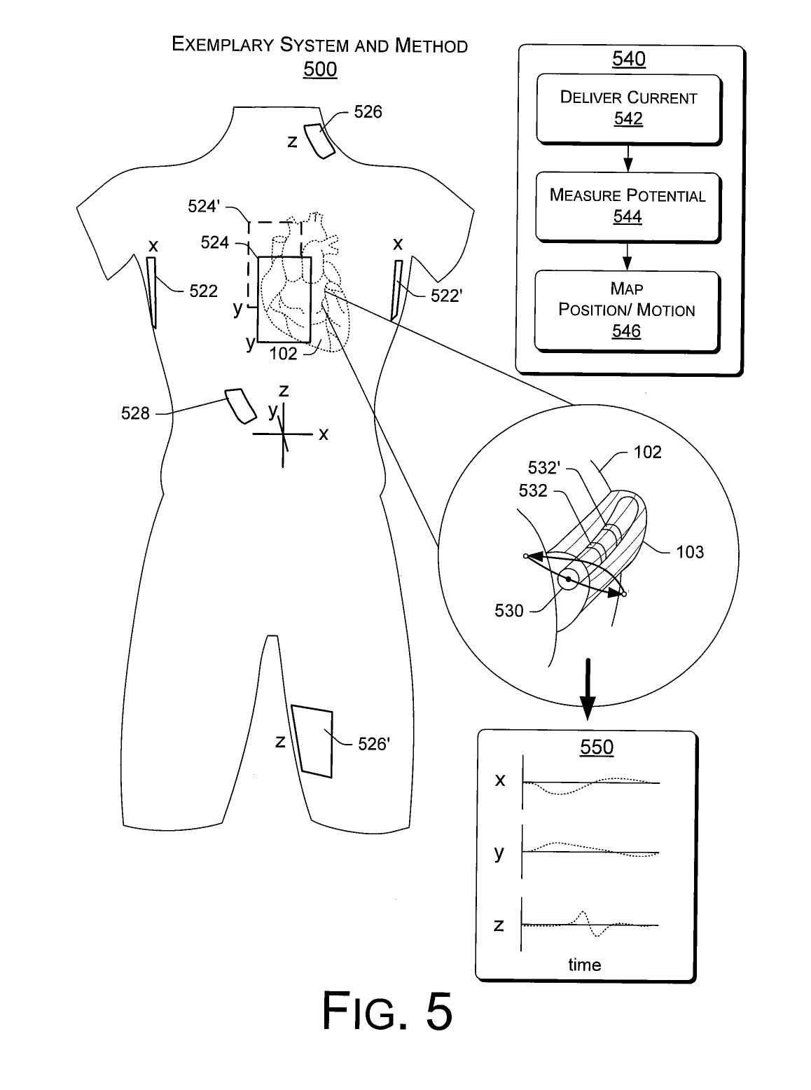

FIG. 5 shows an arrangement and method 500 that may rely in part on a commercially available system marketed as ENSITE® NAVX® navigation and visualization system (see also LocaLisa system). The ENSITE® NAVX® system is a computerized storage and display system for use in electrophysiology studies of the human heart. The system consists of a console workstation, patient interface unit, and an electrophysiology mapping catheter and/or surface electrode kit. By visualizing the global activation pattern seen on color-coded isopotential maps in the system, in conjunction with the reconstructed electrograms, an electrophysiologist can identify the source of an arrhythmia and can navigate to a defined area for therapy. The ENSITE® system is also useful in treating patients with simpler arrhythmias by providing non-fluoroscopic navigation and visualization of conventional electrophysiology (EP) catheters.

As shown in FIG. 5, electrodes 532, 532′, which may be part of a standard EP catheter 530 (or lead), sense electrical potential associated with current signals transmitted between three pairs of surface electrode patches 522, 522′ (x-axis), 524, 524′ (y-axis) and 526, 526′ (z-axis). An additional electrode patch 528 is available for reference, grounding or other function. The ENSITE® NAVX® System can also collect electrical data from a catheter and can plot a cardiac electrogram 570 from a particular location (e.g., cardiac vein 103 of heart 102). Information acquired may be displayed as a 3-D isopotential map and as virtual electrograms. Repositioning of the catheter allows for plotting of cardiac electrograms from other locations. Multiple catheters may be used as well. A cardiac electrogram or electrocardiogram (ECG) of normal heart activity (e.g., polarization, depolarization, etc.) typically shows atrial depolarization as a “P wave”, ventricular depolarization as an “R wave”, or QRS complex, and repolarization as a “T wave”. The ENSITE® NAVX® system may use electrical information to track or navigate movement and construct three-dimensional (3-D) models of a chamber of the heart.

A clinician can use the ENSITE® NAVX® system to create a 3-D model of a chamber in the heart for purposes of treating arrhythmia (e.g., treatment via tissue ablation). To create the 3-D model, the clinician applies surface patches to the body. The ENSITE® NAVX® system transmits an electrical signal between the patches and the system then senses the electrical signal using one or more catheters positioned in the body. The clinician may sweep a catheter with electrodes across a chamber of the heart to outline structure. Signals acquired during the sweep, associated with various positions, can then be used to generate a 3-D model. A display can display a diagram of heart morphology, which, in turn, may help guide an ablation catheter to a point for tissue ablation.

With respect to the foregoing discussion of current delivery and potential measurement, per a method 540, a system (e.g., such as the ENSITE® NAVX® system) delivers low level separable currents from the three substantially orthogonal electrode pairs (522, 522′, 524, 524′, 526, 526′) positioned on the body surface (delivery block 542) and optionally the electrode 528 (or one or more other electrodes). The specific position of a catheter (or lead) electrode within a chamber of the heart can then be established based on three resulting potentials measured between the recording electrode with respect to a reference electrode, as seen over the distance from each patch set to the recording tip electrode (measurement block 544). Sequential positioning of a catheter (or lead) at multiple sites along the endocardial surface of a specific chamber can establish that chamber's geometry, i.e., position mapping (position/motion mapping block 546). Where the catheter (or lead) 530 moves, the method 540 may also measure motion.

In addition to mapping at specific points, the ENSITE® NAVX® system provides for interpolation (mapping a smooth surface) onto which activation voltages and times can be registered. Around 50 points are required to establish a surface geometry and activation of a chamber at an appropriate resolution. The ENSITE® NAVX® system also permits the simultaneous display of multiple catheter electrode sites, and also reflects real-time motion of both ablation catheters and those positioned elsewhere in the heart.

The ENSITE® NAVX® system relies on catheters for temporary placement in the body. Various exemplary techniques described herein optionally use one or more electrodes for chronic implantation. Such electrodes may be associated with a lead, an implantable device, or other chronically implantable component. Referring again to FIG. 3, the configuration block 310 indicates that intraoperative configurations 312 and chronic configurations 314 may be available. Intraoperative configurations 312 may rely on a catheter and/or a lead suitable for chronic implantation.

With respect to motion, the exemplary system and method 500 may track motion of an electrode in one or more dimensions. For example, a plot 550 of motion versus time for three dimensions corresponds to motion of one or more electrodes of the catheter (or lead) 530 positioned in a vessel 103 of the heart 102 where the catheter (or lead) 530 includes the one or more electrodes 532, 532′. Two arrows indicate possible motion of the catheter (or lead) 530 where hysteresis may occur over a cardiac cycle. For example, a systolic path may differ from a diastolic path. An exemplary method may analyze hysteresis for any of a variety of purposes including selection of a stimulation site, selection of a sensing site, diagnosis of cardiac condition, etc.

The exemplary method 540, as mentioned, includes the delivery block 542 for delivery of current, the measurement block 544 to measure potential in a field defined by the delivered current and the mapping block 546 to map motion based at least in part on the measured potential. According to such a method, motion during systole and/or diastole may be associated with physiological information. Alone, or in combination with physiological information, the motion information may be used for selection of optimal stimulation site(s), determination of hemodynamic surrogates (e.g., surrogates to stroke volume, contractility, etc.), optimization of CRT, placement of leads, determination of pacing parameters (AV delay, VV delay, etc.), identification of locations for ablation, etc.

The system 500 may use one or more features of the aforementioned ENSITE® NAVX® system. For example, one or more pairs of electrodes (522, 522′, 524, 524′, 526, 526′) may be used to define one or more dimensions by delivering an electrical signal or signals to a body and/or by sensing an electrical signal or signals. Such electrodes (e.g., patch electrodes) may be used in conjunction with one or more electrodes positioned in the body (e.g., the electrodes 532, 532′).

The exemplary system 500 may be used to track motion of one or more electrodes due to systolic motion, diastolic motion, respiratory motion, etc. Electrodes may be positioned along the endocardium and/or epicardium during a scouting or mapping process for use in conjunction with acquiring position information and/or physiological information. Such information may also be used to identify the optimal location of an electrode or electrodes for use in delivering CRT. For example, a location may be selected for optimal stimulation, for optimal sensing, or other purposes (e.g., anchoring ability, etc.).

With respect to stimulation, stimulation may be delivered to control cardiac mechanics (e.g., contraction of a chamber of the heart) and position information may be acquired where the position information is associated with the controlled cardiac mechanics. An exemplary selection process may identify the best stimulation site based on factors such as electrical activity, electromechanical delay, extent of motion, synchronicity of motion where motion may be classified as systolic motion or diastolic motion. In general, position information corresponds to position of an electrode or electrodes (e.g., endocardial electrodes, epicardial electrodes, etc.) with respect to time and may be related to motion of the heart.

FIG. 6 shows an exemplary arrangement 600 with respect to a surface rendering and two cross-sectional views of the heart 102 along with two electrode-bearing leads 105, 107, a plot 605 of electrode displacement with respect to time and various electrograms 607.

The surface view of the heart 102 shows an occluded artery that may be the cause of a scar 109, which is shown in the two cross-sectional views of the heart 102. As indicated, the lead 105 is positioned in the right ventricle while the lead 107 is positioned along a surface of the left ventricle. The scar 109 is surrounded by a border zone and a remote zone, which may be expected to have or exhibit characteristics that differ from the scar 109 and that may depend on distance from the scar 109. The plot 605 shows six hypothetical curves that correspond to electrodes LV-Tip and LV1-5 of the lead 107. For example, the lower curve has the least displacement with respect to time and may correspond to motion of the LV-Tip electrode as it is proximate to the scar 109. In contrast, the highest curve has the most displacement with respect to time and may correspond to motion of the LV1 electrode as it is the furthest from the scar 109 of the electrodes of the lead 107.

To locate damaged or otherwise compromised tissue, various exemplary methods use cardiac electrograms. A cardiac electrogram may be acquired using electrodes implanted in the body (e.g., subcutaneous, intracardiac, etc.) and/or so-called surface electrodes (e.g., cutaneous electrodes, etc.). In general, a cardiac electrogram acquired using one or more of the former types of electrodes is labeled an EGM while a cardiac electrogram acquired using solely the latter type of electrodes is labeled an ECG. The former group, i.e., EGM, include intracardiac electrograms (IEGMs). In either instance, a cardiac electrogram typically exhibits certain standard features such as a P wave, an R wave, an S wave, a Q wave, a T wave, a QRS complex, etc. Where contraction of a chamber of the heart occurs responsive to delivery of an electrical stimulus, then the electrical waveform may be considered an evoked response (ER) and labeled an A wave, a V wave, etc., depending on the chamber, or chambers, stimulated. Also, an IEGM can include information to determine pacing latency, generally defined as the difference between the delivery time of an electrical stimulus and the time an ER commences. In some instances, pacing latency may be defined on another basis, for example, based on a minimum in amplitude for an ER, maximum slope of an ER, etc., as used by an ER detection algorithm.

Various studies have related cardiac electrograms to damage. For example, subendocardial ischemia can prolong local recovery time. Since repolarization normally proceeds in an epicardial-to-endocardial direction, delayed recovery in the subendocardial region due to ischemia does not reverse the direction of repolarization but merely lengthens it. This generally results in a prolonged QT interval or increased amplitude of the T wave or both as recorded by the electrodes overlying, or otherwise sensing activity at, the subendocardial ischemic region.

Subepicardial or transmural ischemia is typically said to exist when ischemia extends subepicardially. This type of damage has a more visible effect on recovery of subepicardial cells compared with subendocardial cells. Recovery is more delayed in the subepicardial layers, and the subendocardial muscle fibers often seem to recover first. Repolarization is endocardial-to-epicardial, resulting in inversion of the T waves in leads overlying, or otherwise sensing activity at, the ischemic regions.

Injury to myocardial cells results when an ischemic process is more severe. Subendocardial injury on a surface ECG (i.e., an ECG) is typically manifested by ST segment depression while, in contrast, subepicardial or transmural injury is manifested as ST segment elevation. In patients with coronary artery disease, ischemia, injury and myocardial infarction of different areas can coexist and produce mixed and complex ECG patterns.

The term infarction describes necrosis or death of myocardial cells. Atherosclerotic heart disease is the most common underlying cause of myocardial infarction. The left ventricle is the predominant site for infarction; however, right ventricular infarction occasionally coexists with infarction of the inferior wall of the left ventricle. The appearance of pathological Q waves is the most characteristic ECG finding of transmural myocardial infarction of the left ventricle. A pathological Q wave is defined as an initial downward deflection of a duration of about 40 ms or more in any lead of a multi-lead surface ECG unit (except lead III and lead aVR). The Q wave appears when the infarcted muscle is electrically inert and the loss of forces normally generated by the infarcted area leaves unbalanced forces of variable magnitude in the opposite direction from a remote region or zone (e.g., an opposite wall). These forces can be represented by a vector directed away from the site of infarction and seen as a negative wave (Q wave) by electrodes overlying, or otherwise sensing activity at, the infarcted region.

During acute myocardial infarction, the central area of necrosis is generally surrounded by an area of injury, which in turn is surrounded by an area of ischemia. Thus, various stages of myocardial damage can coexist. One commonly used distinction between ischemia and necrosis is whether the phenomenon is reversible. Transient myocardial ischemia that produces T wave, and sometimes ST segment abnormalities, can be reversible without producing permanent damage and is not accompanied by serum enzyme elevation.

Two types of myocardial infarction can be typically observed electrocardiographically: Q wave infarction and Non-Q wave infarction. Q wave infarction, which is diagnosed by the presence of pathological Q waves and is also called transmural infarction. However, transmural infarction is not always present, hence, the term Q wave infarction may be preferable for ECG description. Non-Q wave infarction is typically diagnosed based on the presence of ST depression and T wave abnormalities. Elevation of serum enzymes is expected in both types of infarction. In the absence of enzyme elevation, ST and T wave abnormalities are interpreted usually as due to injury or ischemia rather than infarction.

As already mentioned, a damage site (e.g., ischemia, injury, infarction) can be localized to some extent using cardiac electrograms, for example, the general location of an infarct can be detected by an analysis of a 12-lead ECG. Leads that best detect changes in commonly described locations are classified as follows: Inferior (or diaphragmatic) wall—II, II and aVF; Septal—V1 and V2; Anteroseptal—V1, V2, V3 and sometimes V4; Anterior—V3, V4 and sometimes V2; Apical—V3, V4 or both; Lateral—I, aVL, V5 and V6; and Extensive anterior—I, aVL and V1 through V6.

Posterior wall infarction does not typically produce Q wave abnormalities in conventional leads and is generally diagnosed in the presence of tall R waves in V1 and V2. The classic changes of necrosis (Q waves), injury (ST elevation), and ischemia (T wave inversion) may all be seen during acute infarction. In recovery, the ST segment is the earliest change that normalizes, then the T wave; the Q wave usually persists. Therefore, the age of the infarction can be roughly estimated from the appearance of the ST segment and T wave. The presence of the Q wave in the absence of ST and T wave abnormality generally indicates prior or healed infarction. Although the presence of a Q wave with a 40 ms duration is usually sufficient for diagnosis, criteria defining the abnormal depth of Q waves in various leads have been established. For example, in lead I, the abnormal Q wave must be more than 10 percent of QRS amplitude; in leads II and aVF, it should exceed 25 percent; and in aVL it should equal 50 percent of R wave amplitude. Q waves in V2 through V6 are typically considered abnormal if greater than 25 percent of R wave amplitude.

A deep Q wave generally indicates myocardial necrosis, although similar patterns may be produced by other conditions, such as WPW syndrome, connected transportation of the great vessels, etc. ST segment elevation can be observed in conditions other than acute myocardial infarction.

With respect to ST segment elevation, other causes of ST segment elevation include the following: acute pericarditis (ST elevation in acute pericarditis is generally diffuse and does not follow the pattern of blood supply. As a rule these changes are not accompanied by reciprocal depression of the ST segment in other leads); early repolarization (In some patients without known heart disease, particularly young patients, early takeoff of the ST segment may be seen); ventricular aneurysm (After acute myocardial infarction, the ST segment usually normalizes. However, in the presence of a persistent aneurysm in the region of infarction, ST segment elevation may persist indefinitely).

Abnormal T waves can be seen in a variety of conditions other than myocardial ischemia, including: hyperventilation, cerebrovascular disease, mitral valve prolapse, right or left ventricular hypertrophy, conduction abnormalities (right or left bundle branch block), ventricular preexcitation, myocarditis, electrolyte imbalance, cardioactive drugs such as digitalis and antiarrhythmic agents, or for no obvious cause (particularly in women). Thus, cardiac electrograms may provide insight into location, severity, age, repair, etc., of myocardial tissue damage (e.g., ischemia, injury and/or infarct).

FIG. 6 shows cardiac infarct along with a series of cardiac electrograms 607 for the remote zone, the border zone and the scar zone. The remote zone cardiac electrogram (leftmost electrogram) exhibits a depressed ST segment and may represent an ischemic or injured region. The border zone cardiac electrogram (middle electrogram) exhibits an elevated ST segment and a prolonged PR segment and may represent subepicardial or transmural injury. The infarct zone cardiac electrogram (rightmost electrogram) exhibits a deep Q wave, which generally indicates myocardial necrosis, i.e., infarct.

Such information may be acquired from patient populations (e.g., prior infarct, heart failure, normal, young, old, etc.) and used for purposes of analyzing electrical information for a particular patient. For example, electrical information for healthy patients may be used to establish one or more standard segments (e.g., standard time for ST segment, standard amplitude for ST, Q, PR, etc.). One or more of such standards may then be used to assess cardiac condition of a particular patient. In a specific example, PR and ST interval times are acquired for a patient and compared to standard PR and ST interval times. The comparison may be a ratio based comparison (e.g., PR/ST, ST/PR, etc.), a percentage based comparison, etc., where the comparison can help assess a region of the patient's heart with respect to an infarct (e.g., distance of region from an infarct zone, damage level, etc.).

Various exemplary methods include acquiring one or more cardiac electrograms and analyzing the one or more cardiac electrograms to determine health of a myocardial region and/or to locate a border between types of myocardial tissue (i.e., border between infarct and injury, injury and ischemic, ischemic and healthy or normal for patient, etc.).

As described herein, an exemplary method or system may map information from local cardiac electrograms. For example, a method or system may generate a ST segment elevation map (e.g., as evidence of subepicardial or transmural injury), a ST segment depression map (e.g., as evidence of subendocardial injury), a PR segment prolongation map (e.g., as evidence of subepicardial or transmural injury) a PR segment depression map (e.g., as evidence of subepicardial atrial injury or acute pericarditis). Information from local cardiac electrograms may be combined with other information to generate a composite map, for example, to more accurately identify a damaged region of the heart. Such information may aid in discrimination of ventricular damage and atrial damage.

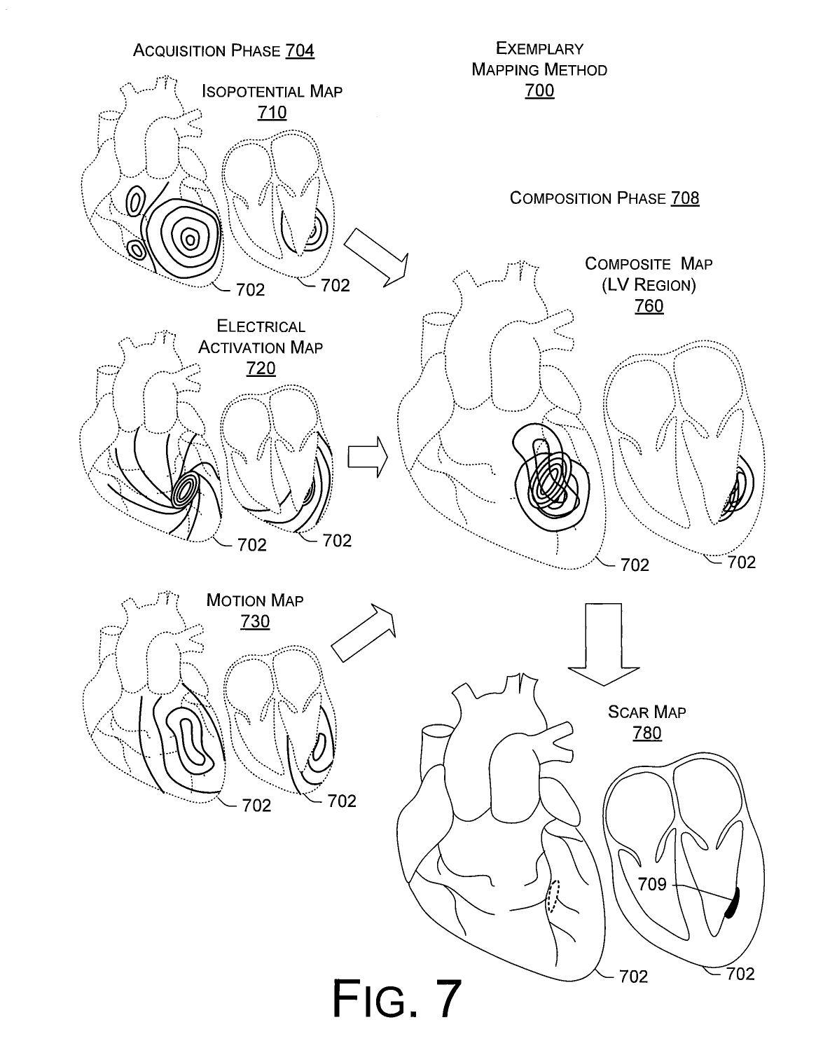

FIG. 7 shows an exemplary mapping method 700 with respect to various surface and cross-sectional views of the heart 702. The exemplary method 700 includes an acquisition phase 704 and a composition phase 708. The acquisition phase 704 acquires data associated with position information and the composition phase 708 composes one or more composite maps based on the acquired data.

In the example of FIG. 7, the acquisition phase 704 acquires isopotential data as indicated by an isopotential map 710, electrical activation data as indicated by an electrical activation map 720 (e.g., isochrones) and motion data as indicated by a motion map 730 (e.g., isomotions). The maps 710, 720 and 730 are approximate and shown to demonstrate some aspects of isopotentials, isochrones and isomotions. In the map 710, the isopotentials form a “bulls-eye” around a region of low potential (e.g., peak-to-peak for a QRS complex). The effected region may be a ring or a portion of a ring in the wall of the left ventricle with varying degree of damage and hence potential differences.

In the map 720, the isochrones essentially form concentric contours around a region with little or no signs of electrical activation. In this example, if the sampling window for the data is around one second then the longest electrical activation time assigned would also be around one second. In general, for a healthy heart, the PR interval is around 120 ms to about 200 ms such that most of the myocardium would exhibit electrical activation at no more than around 300 ms (from initiation of a sinus stimulus). Thus, damaged tissue may be expected to include contours from greater than 300 ms to the end of the sampling interval. If contours are plotted for increments of 20 ms, each 100 ms would include 5 contours. If the sampling interval is about 1 second and the PR interval about 200 ms then a scar may be surrounded by about 40 contours (assuming increments of about 20 ms between each contour).

In the map 730, the isomotion contours aim to identify a region of low motion or abnormal motion. As mentioned, motion may account for direction and/or motion or direction at a particular time or period of time (e.g., systolic, diastolic). Thus, where damaged tissue bulges outward during contraction of the left ventricle, the direction may be noted and displayed in the map 730 (e.g., via shading, coloring, hatching, dashed lines, etc.).

In the example of FIG. 7, the composition phase 708 composes a composite map 760 for a left ventricular region. For example, the composition phase 708 may determine based on one or more criteria that certain acquired information is more relevant than other acquired information. Specifically, for the isopotential data, the composition phase 708 may select data that represents the lowest 20% of the isopotentials, which may be indicative of damaged or scarred tissue; for the electrical activation data, the composition phase 708 may select data that represents the longest 10% of electrical activation times, which may be indicative of damaged or scarred tissue; and, for the motion data, the composition phase 708 may select data that represents the smallest 15% of motion, which may be indicative of damaged or scarred tissue. Thus, the composite map 760 need not necessarily include or display all of the data of each individual map 710, 720 or 730.

Given the composite map 760, or the data underlying the composite map 760, the composition phase 708 identifies a damaged or scarred region 709, which is shown in a “scar” map 780. As the scar 709 does not extend to the surface of the heart 702, which is shown as a filled region in the cross-sectional view of the heart 702, a dashed line outlines the scar 709 in the surface rendering of the heart 702.

As described herein, the composition phase 708 aims to define a damaged or scarred region to with a certain degree of probability using multiple metrics, each of which is associated with position information. Hence, the scar 709 may be identified by overlaying contours or by weighting metrics using an equation to determine a region with, for example, the highest or the lowest value.

As described herein, an exemplary equation for determining a contour or contours for a damaged or a scarred region (Contours-DR) may be as follows:

Contours-DR=w_{IPM}*[Contours X_{IPM}]+w_{EAM}*[Contours X_{EAM}]+w_{MM}*[Contours X_{MM}]

In this equation, contours are selected based on a percentile or value based criterion or criteria (e.g., on a factor “X” such as 20%, 10% and 15%) and the contours for the certain isopotential data, electrical activation data and the motion data are weighted by the individual weights w_{IPM}, w_{EAM}, and w_{MM}. In this example, 3-D contour data may be available for each of the metrics where the selection and the weighting occur while maintaining the position information of the respective contours. One or more resulting contours can be drawn as composite contours to identify a damaged or scarred region.

For example, for a given point of overlap of an isopotential contour that corresponds to 10% of a maximum potential (i.e., low potential is “bad”), an isochrone that corresponds to 60% of a cardiac cycle (i.e., 100% of cardiac cycle is the “worst”), a motion contour that corresponds to 10% of maximum motion (i.e., no motion is “bad”), the equation may aim to equate low values with damage or scarring. To accomplish this task, the isochrone can be reformulated such that a long time (e.g., approaching the duration of a cardiac cycle) results in a small value. For example, 100%−60%=40% such that the later the activation, the smaller the value. Next, weights may be applied where w_{IPM}=0.5; w_{EAM}=0.1 and w_{MM}=1.2. For the given point, the resulting value would be: 0.5*0.1+0.1*0.4+1.2*0.1=0.21. Weights may be selected to normalize the metrics and/or, for example, to cause one metric to contribute more or less to the resulting value.

Scaling, weighting, etc., may occur through user selections or automatically based on one or more criteria. With respect to display of a map, an exemplary method may rely on RGB or other color scheme to overlap data (e.g., contour bounded regions) to thereby indicate a damaged or a scarred region.

As described herein, an exemplary equation for determining a centroid for a damaged or a scarred region (Centroid-DR) may be as follows:

Centroid-DR=w_{IPM}*[Centroid X_{IPM}]+w_{EAM}*[Centroid X_{EAM}]+w_{MM}*[Centroid X_{MM}]

In this equation, centroids are calculated for certain data (e.g., percentile or value based on a factor “X” such as 20%, 10% and 15%) and the centroids for the certain isopotential data, electrical activation data and the motion data are weighted by the individual weights W_{IPM}, W_{EAM}, and w_{MM}. In this example, 3-D contour data may be available for each of the metrics and a volumetric centroid calculated.

Depending on the shape of a damaged region or scarred region, a centroid may lie outside the actually myocardium. For example, consider a damaged annular section of myocardium spanning about 60 degrees about a long axis of the left ventricle. In this example, the centroid may lie in the space defined by the ventricular wall. Space transforms can be optionally used to avoid such a result. For example, a section of a ventricular wall may be transformed to a flat sheet. In this example, a centroid may be calculated as indicative of a damaged region. A reverse transform may then be applied that maintains the centroid of the damage region within the ventricular wall.

In some instances, the centroid of a “solid” volume is the same as the center of mass. Where particular variations in the density of the volume are known or other properties, an associated centroid may be calculated. For example, a centroid may be based solely on volume for contours or it may be calculated based on local metric values within the volume. In the former, an exemplary method may select an isopotential contour of a certain value and then calculate a centroid based on the volume bounded by the isopotential contour. In the latter, isopotential values within the bounds of the particular isopotential contour may be used akin to density values for a center of mass calculation. The latter provides a center that may be located closer to actual lower potential values bounded by the particular contour and hence more accurately represent a point in a region that is damaged or scarred when compared to a centroid based on an isopotential contour bounded volume alone.

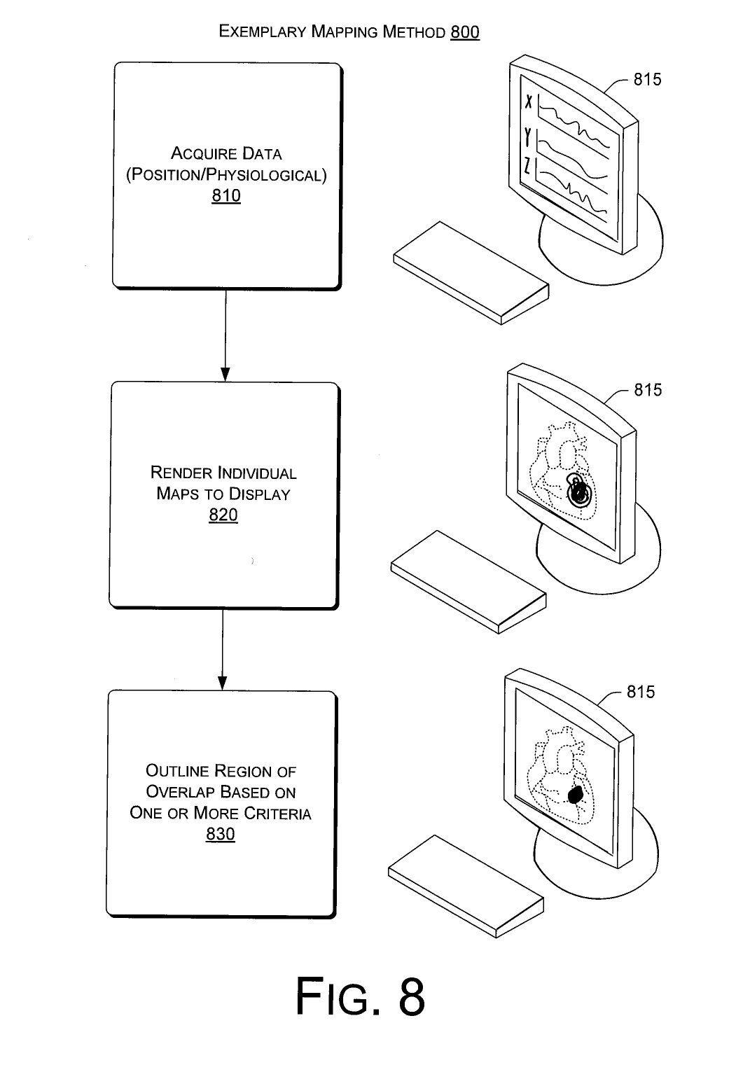

FIG. 8 shows an exemplary mapping method 800 along with an exemplary display system 815. In an acquisition block 810, the method 800 acquires data where the data specifies positions associated with physiological data and positions with respect to time (e.g., motion information). The display system 815 may render acquired data as indicated along with coordinates or other information. In a rendition block 820, the method 800 renders individual data maps or layers to a display. In the example of FIG. 8, the display system 815 shows an outline of the heart along with contours for three different metrics: isopotential, isochronal and isomotion. The data for each map may be stored in memory (e.g., display buffer) where data for each metric may be stored as a separate layer that can be manipulated individually and separately from data for other metrics.

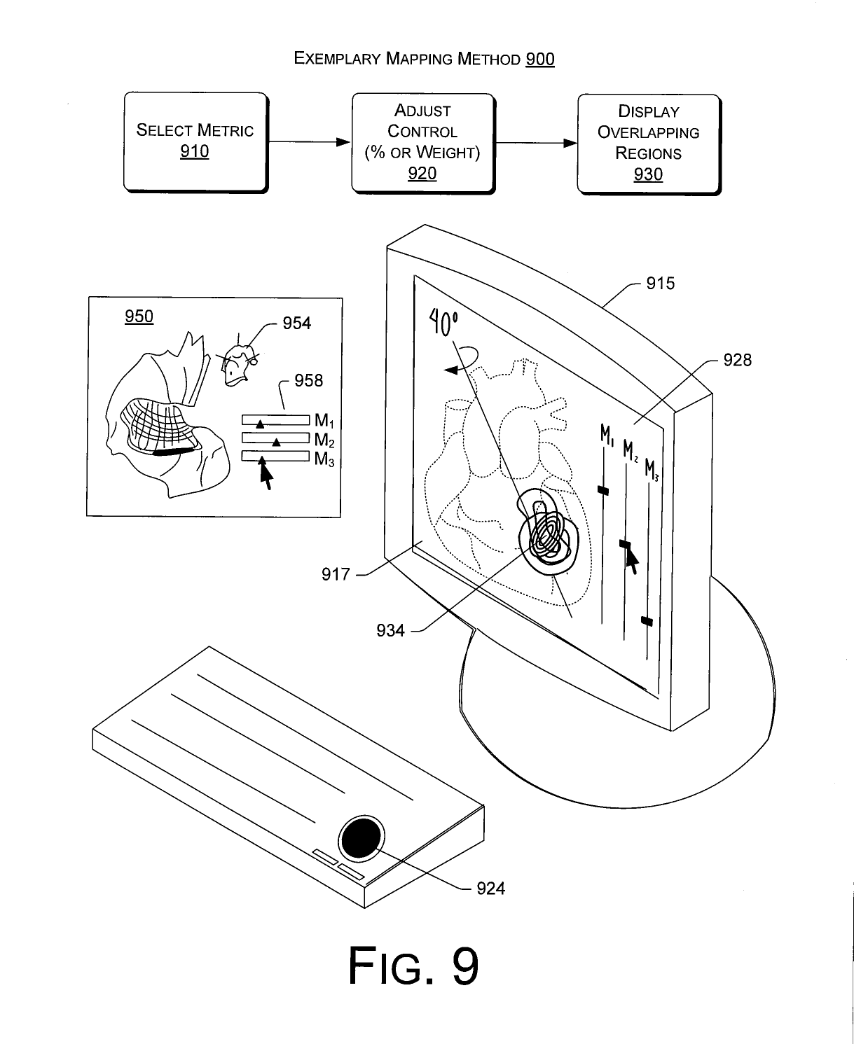

FIG. 9 shows an exemplary mapping method 900 that allows a user to adjust one or more parameters for mapping metrics and rendering the metrics with respect to anatomical features of the heart. The method 900 includes a selection block 910 for selecting a metric. For example, a display system 915 may display controls that allow for selection of metric (e.g., M₁, M₂, M₃). An adjustment block 920 allows the user to adjust one or more parameters for the selected metric. For example, the user may adjust a parameter that determines the increment between contours, the minimum contour, the maximum contour, a weight for a contour, a transparency for a contour, a color for a contour, a shading or fill for a contour, etc.

In the example of FIG. 9, the display system 915 shows a pointing mechanism 924 that can be manipulated to control a user interface 917, for example, to individually adjust three slider controls 928 (M₁, M₂, M₃) or to rotate a view about an axis (e.g., long axis of the left ventricle). Upon adjustment, a display block 930 displays overlapping regions for the metrics. Overall, the method 900 allows a user to adjust how data is displayed for various metrics to understand better cardiac health, particularly whether or where a damaged or a scar region may exist.

FIG. 9 shows another user interface 950 that displays a human torso with respect to a coordinate system and controls 958 for various metrics (e.g., M₁, M₂, M₃). In this particular example, a cutaway view of the heart is shown to expose a chamber and a wall. According to the method 900, a user may select a metric 910 and adjust a control 920 to instruct a system to display overlapping regions 930, which can indicate presence of damaged or scarred tissue (see, e.g., filled region of wall).

In various examples, an input device is shown along with a monitor or display. It is understood that various mechanisms exist to allow for communication between the input device and the display (e.g., wired or wireless). Further, the input device and display may connect via wire or wirelessly to a system such as the ENSITE® localization system. The input device and display may optionally include memory and one or more processors (e.g., integral computing device) suitable to execute one or more modules to perform various methods described herein. In general, system for handling three-dimensional data and rendering views of such data (e.g., as maps) typically include rich graphics processing capabilities such as one or more graphical processing units (GPUs).

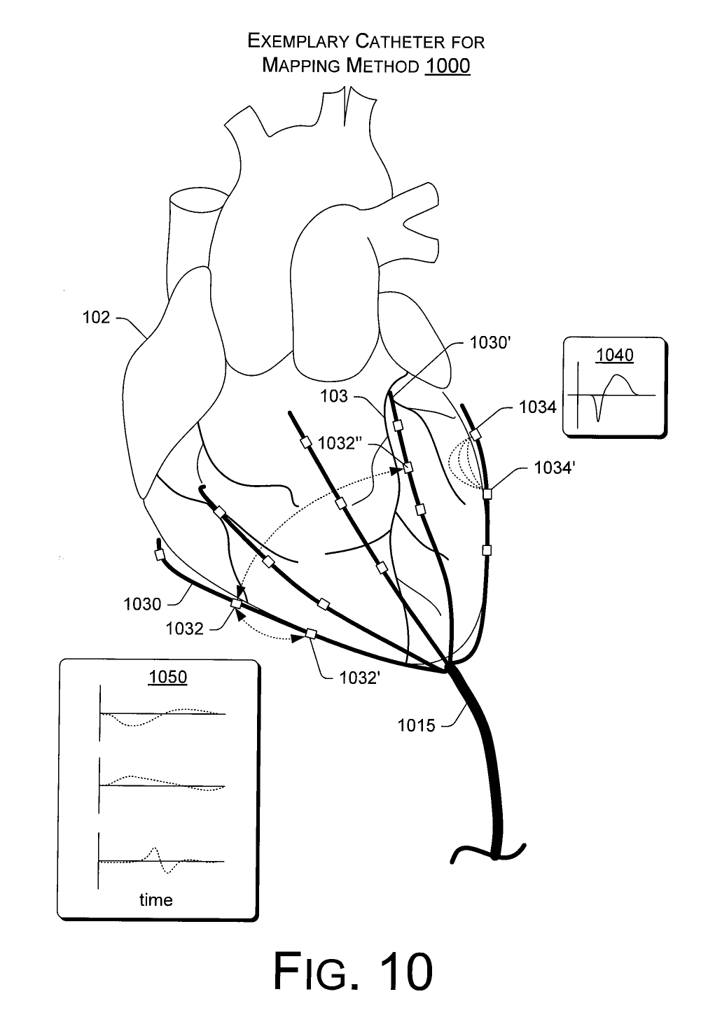

FIG. 10 shows an exemplary catheter 1000 for use in acquiring physiological information 1040 and/or position information 1050. The catheter 1000 includes a main branch 1015 that branches into a plurality of splines 1030, 1030′ where each spline may include one or more electrodes 1032, 1032′, 1032″, 1034, 1034′. Noting that not all splines or electrodes include reference numerals in FIG. 10. Further, while not shown in FIG. 10, the catheter 1000 includes one or more connectors to electrically connect the various electrodes to an acquisition device or system (e.g., a computer-based data acquisition system). The catheter 1000 may operate in conjunction with one or more other electrodes not shown in FIG. 10. For example, the main branch 1015 may include a reference electrode and/or one or more surface electrodes may be used. The catheter 1000 may be used in conjunction with the system and method of FIG. 5 where, for example, current is introduced using surface electrodes 522, 522′, 524, 524′, 526 and 526′.

While the example of FIG. 10 refers to a catheter, in an alternative system, the catheter 1000 may be a lead configured for chronic implantation in the body and with appropriate features for electrical connection to an implantable device. In yet another alternative, the lead includes appropriate electronics and a power supply disposed along one or more sections of the lead. In this latter example, a separate implantable device may not be required.

The basket-like catheter 1000 (or alternative lead) may be introduced into the body via any of a variety of procedures. For example, such a catheter may be positioned using subxyphoid access to the pericardium. Such a technique may use fluoroscopic guidance. A retractable sheath may be used to expose splines or splines may extend out of a sheath. The splines may have some resiliency such that the splines fit snugly to the myocardial surface. The splines may include one or more anchoring mechanisms to help anchor the splines. Such mechanisms may be extendable and/or retractable. In general, such mechanisms avoid risk of rupture to cardiac arteries. Fluoroscopic or other guidance may be used to minimize risk of injury to one or more cardiac arteries.

While anterior splines are shown in FIG. 10, the catheter 1000 may include posterior splines as well. The splines 1030, 1030′ of the catheter 1000 are capable of surrounding a portion of the ventricles. The splines 1030 may be positioned across one or more vessels such as cardiac veins 103. Such veins 103 may be of sufficient size to allow for placement of an electrode via the coronary sinus or other venous access. In general, for purposes of CRT, an electrode may be positioned via a vessel or via pericardial access.

As already mentioned, the catheter 1000 may be used in conjunction with one or more patch electrodes positioned on the surface of a patient's body. In such an arrangement, various electrodes of the catheter 1000 may be used to measure potential or to deliver current and various patch electrodes may be used to deliver current or to measure current (see, e.g., system and method 500 of FIG. 5). For example, the patch electrodes may deliver current while the catheter electrodes measure potential. Referring to FIG. 10, cardiac mechanics will cause movement of the splines 1030 and associated electrodes 1032, 1032′, 1032″, 1034, 1034′. In turn, the measured potential will vary as a function of cardiac mechanics.

Potential may be measured across any of the electrodes of the catheter 1000. For example, potential may be measured between the electrode 1032 and the electrode 1032′ (e.g., same spline) or between the electrode 1032 and the electrode 1032″ (e.g., different splines). Accordingly, using a catheter with multiple electrodes positioned in the pericardial space, a variety of measurements may be made to understand better cardiac health.

While the catheter 1000 may be used for acquiring motion information, one or more of the electrodes 1032, 1032′, 1032″, 1034, 1034′ may be used to deliver stimulation energy to the myocardium. For example, the electrodes 1034, 1034′ may be used to deliver stimulation energy to the left ventricle (e.g., lateral wall of left ventricle) at a time and level sufficient to cause an evoked response 1040. After delivery of stimulation energy, either or both of the electrodes 1034, 1034′ may be used to measure potential over time, which, in turn, may be used to determine motion of the lateral wall of the left ventricle when stimulated at a stimulation site defined by the electrodes 1034, 1034′. Cardiac electrical activity information 1040 may be used in conjunction with motion information 1650 for any of a variety of purposes.

Various studies indicate that fat pads or neural plexuses exist on the epicardial surface. Where a therapy includes delivery of energy to a nerve (e.g. a fat pad, an autonomic nerve, neural plexus, etc.), then the catheter 1000 may be used to help identify an appropriate stimulation site or delivery of energy to such a site may occur in conjunction with acquisition of motion information. Another catheter, lead, electrode, etc., may be used to deliver energy to a nerve where the catheter 1000 acquires motion information, for example, as a function of such energy delivery. Further, a clinician may administer a drug, a maneuver (Valsalva maneuver, tilt test, etc.), etc., that could affect cardiac performance where the catheter 1000 is used to acquire position or motion information as a function of such action.

An exemplary catheter includes a sheath, a plurality of splines extending from the sheath and configured to conform to the heart, a plurality of electrodes disposed on various splines and a connector to connect the electrodes to a measuring device to measure potentials using the electrodes. Such a catheter may include current delivery electrodes and a connector to connect the current delivery electrodes to a current delivery device. In such an example, the measuring device measures potentials associated with the current delivered by the current delivery electrodes. Further, the measuring device and the current delivery device may be the same device.

In an exemplary mapping method, a patient may have a basket catheter (e.g., the catheter 1000 of FIG. 10 or a modified interchamber basket catheter such as the CONSTELLATION® catheter marketed by Boston Scientific, Natick, Mass. having an open end), which has multiple splines spanning the circumference of the chamber, placed in the intrapericardial space, over at least a portion of the heart (e.g., including a portion of the LV chamber). Such a catheter may include splines that are compliant and deform with the contraction and relaxation of the heart during a cardiac cycle to thereby capture motion of the myocardium. While two basket types of catheters have been mentioned, alternatively, a balloon catheter having multiple splines may be inserted into LV chamber via retrograde aortic access.

As described herein, an exemplary system includes one or more processors, memory and control logic to acquire myocardial potential data associated with position information, acquire myocardial electrical activation data associated with position information, acquire myocardial position data with respect to time, generate isopotential contours based on the potential data, generate isochronal contours based on the electrical activation data, generate isomotion contours based on the position data with respect to time, and overlay the generated isopotential contours, isochronal contours and isomotion contours on a display to indicate a region of myocardial damage with respect to a map (e.g., a map that can include one or more anatomical markers). In such a system, the control logic to overlay may be configured to relatively weight isopotential contours, isochronal contours and isomotion contours, for example, to increase or decrease their respective contribution to an overlay on a display. Such a feature can allow the system or a clinician to more accurately indicate a region of myocardial damage with respect to a map.

As described herein, an exemplary system can include control logic to render adjustable controls to a display to individually weight isopotential contours, isochronal contours and isomotion contours to increase or decrease their respective contribution to an overlay on the display. Such a feature can allow the system or a clinician to more accurately indicate a region of myocardial damage with respect to a map.

As described herein, an exemplary system can include control logic to select only isopotential contours that have values less than a predetermined value, for example, to more accurately indicate a region of myocardial damage. Such a predetermined value may be referred to as a criterion for a measure of cardiac performance. Similarly, an exemplary system can include control logic to select only isochronal contours that have values greater than a predetermined value, to select only isomotion contours that comprise values less than a predetermined value, etc.

An exemplary system can include control logic to select only some of contours based on one or more predetermined values and to weight the selected contours to increase or decrease their respective contribution to an overlay on a display, for example, to more accurately indicate a region of myocardial damage.

An exemplary system can include control logic to automatically or by user input indicate myocardial damage by outlining a scar region with respect to a map (e.g., a map that includes anatomical markers).

As described herein, an exemplary system can include control logic to acquire fractionation data associated with position information, generate isofractionation metric contours based on the fractionation data and overlay the generated isofractionation metric contours and the generated isomotion contours on a display, for example, to indicate a region of myocardial damage with respect to a map (e.g., that includes one or more anatomical markers). An exemplary system may include control logic to acquire dominant frequency data associated with position information, generate isofrequency contours based on the dominant frequency data and overlay the generated isofrequency contours and the generated isomotion contours on a display, for example, to indicate a region of myocardial damage with respect to a map (e.g., that includes one or more anatomical markers).

As described herein, an exemplary method includes mapping a first measure of cardiac performance on a map (e.g., that includes one or more anatomical markers); identifying a region on the map as including a myocardial scar; selecting a second measure of cardiac performance; mapping the second measure of cardiac performance on the map; and narrowing the region on the map as including the scar. In such a method, the first measure of cardiac performance may be a cardiac motion measure, a cardiac potential measure or a cardiac timing measure. Similarly, the second measure may be a cardiac motion measure, a cardiac potential measure or a cardiac timing measure.

With respect to narrowing, narrowing may occur by overlaying a contour for a first measure and a contour for a second measure. Such an overlay can define an intersecting region. In a particular example, an intersecting region can have a color caused by mixing a color associated with a contour for a first measure and a different color associated with a contour associated with a second measure. As explained, a method may include more than two measures. For example, a method may include selecting a third measure of cardiac performance; mapping the third measure of cardiac performance on a map; and further narrowing a region on the map as including a scar.

In various examples, mapping of a first measure may create a map that includes isopotential contours. In various examples, mapping of a second measure may create a composite map that includes isopotential and isochronal contours associated with activation of a heart; create a composite map that includes isopotential and isomotion contours associated with activation of a heart; or create a composite map that includes isomotion and isochronal contours associated with activation of a heart.

As mentioned, a measure may be a cardiac potential measure, a cardiac motion measure or a cardiac timing measure. Examples of measures include dominant frequency, fractionation, time to peak displacement, time to peak onset, time to peak slope, ST segment and PR segment; noting that morphologies such as Q wave and T wave morphologies are measures that may be used.

As described herein, an exemplary method may include determining a location for placement of an electrode in a patient's body based on a composite map. Such a method may increase probability of a patient responding to a therapy that relies on the electrode. For example, patients can be classified as having functional or structural or a combination of functional and structural issues that may decrease response to cardiac resynchronization therapy. Composite maps can assist a clinician in placing an electrode with respect to a scarred region of the heart. For example, once a scarred region has been identified on the left ventricle, a clinician may avoid certain veins as candidates for placement of a left ventricular lead.

An exemplary method may include identifying a region as including a scar automatically, for example, responsive to mapping of a first, second or other measure. An exemplary method may include narrowing a region automatically responsive to mapping of a measure. For example, an algorithm may identify an overlap region based on data for two different measures and automatically highlight the overlapped region (e.g., intersecting region) on a display. As mentioned, a method may include providing one or more criterion associated with a measure prior to identifying a region as including a myocardial scar. Similarly, a method may include providing one or more criterion associated with a measure prior to narrowing the region as including the scar.

As described herein, an exemplary system includes one or more processors, memory and control logic to map a first measure of cardiac performance on a map, identify a region on the map as including a myocardial scar, select a second measure of cardiac performance, map the second measure of cardiac performance on the map and narrow the region on the map as including the scar. Such a system may include circuitry configured to acquire potentials from an electrode positioned in a current field and to determine a location for the electrode based on acquired potentials.

An exemplary system may include an input to receive image data for a heart and to map one or more anatomical markers based at least in part on received image data. For example, a system may be configured to receive magnetic resonance image data, X-ray image data, ultrasound image data or a combination thereof. As described herein, an exemplary system may include circuitry configured to acquire electrograms (e.g., IEGMs or surface ECGs).

### Exemplary External Programmer

FIG. 11 illustrates pertinent components of an external programmer 1100 for use in programming an implantable medical device 100 (see, e.g., FIGS. 1 and 2). The external programmer 1100 optionally receives information from other diagnostic equipment 1250, which may be a computing device capable of acquiring motion information related to cardiac mechanics. For example, the equipment 1250 may include a computing device to deliver current and to measure potentials using a variety of electrodes including at least one electrode positionable in the body (e.g., in a vessel, in a chamber of the heart, within the pericardium, etc.). Equipment may include a lead for chronic implantation or a catheter for temporary implantation in a patient's body. Equipment may allow for acquisition of respiratory motion and aid the programmer 1100 in distinguishing respiratory motion from cardiac.

Briefly, the programmer 1100 permits a clinician or other user to program the operation of the implanted device 100 and to retrieve and display information received from the implanted device 100 such as IEGM data and device diagnostic data. Where the device 100 includes a module such as the position detection module 239, then the programmer 1100 may instruct the device 100 to measure potentials and to communicate measured potentials to the programmer via a communication link 1253. The programmer 1100 may also instruct a device or diagnostic equipment to deliver current to generate one or more potential fields within a patient's body where the implantable device 100 may be capable of measuring potentials associated with the field(s).

The external programmer 1100 may be configured to receive and display ECG data from separate external ECG leads 1332 that may be attached to the patient. The programmer 1100 optionally receives ECG information from an ECG unit external to the programmer 1100. As already mentioned, the programmer 1100 may use techniques to account for respiration.

Depending upon the specific programming, the external programmer 1100 may also be capable of processing and analyzing data received from the implanted device 100 and from ECG leads 1332 to, for example, render diagnosis as to medical conditions of the patient or to the operations of the implanted device 100. As noted, the programmer 1100 is also configured to receive data representative of conduction time delays from the atria to the ventricles and to determine, therefrom, an optimal or preferred location for pacing. Further, the programmer 1100 may receive information such as ECG information, IEGM information, information from diagnostic equipment, etc., and determine one or more metric for mapping (e.g., consider the method 300).

Now, considering the components of programmer 1100, operations of the programmer are controlled by a CPU 1302, which may be a generally programmable microprocessor or microcontroller or may be a dedicated processing device such as an application specific integrated circuit (ASIC) or the like. Software instructions to be performed by the CPU are accessed via an internal bus 1304 from a read only memory (ROM) 1306 and random access memory 1330. Additional software may be accessed from a hard drive 1308, floppy drive 1310, and CD ROM drive 1312, or other suitable permanent or removable mass storage device. Depending upon the specific implementation, a basic input output system (BIOS) is retrieved from the ROM 1306 by CPU 1302 at power up. Based upon instructions provided in the BIOS, the CPU 1302 “boots up” the overall system in accordance with well-established computer processing techniques.

Once operating, the CPU 1302 displays a menu of programming options to the user via an LCD display 1214 or other suitable computer display device. To this end, the CPU 1302 may, for example, display a menu of specific programming parameters of the implanted device 100 to be programmed or may display a menu of types of diagnostic data to be retrieved and displayed. In response thereto, the clinician enters various commands via either a touch screen 1216 overlaid on the LCD display or through a standard keyboard 1218 supplemented by additional custom keys 1220, such as an emergency VVI (EVVI) key. The EVVI key sets the implanted device to a safe VVI mode with high pacing outputs. This ensures life sustaining pacing operation in nearly all situations but by no means is it desirable to leave the implantable device in the EVVI mode at all times.

With regard to mapping of metrics, the CPU 1302 includes a 3-D mapping system 1347 and an associated data analysis system 1349, which may be used for weighting, adjusting, etc., for example, as described with respect to FIGS. 7, 8 and 9. The systems 1347 and 1349 may receive position information and physiological information from the implantable device 100 and/or diagnostic equipment 1250. The data analysis system 1349 optionally includes control logic to associate information and to make one or more conclusions based on mapped metrics, for example, as indicated in FIG. 3.

Where information is received from the implanted device 100, a telemetry wand 1328 may be used. Other forms of wireless communication exist as well as forms of communication where the body is used as a “wire” to communicate information from the implantable device 100 to the programmer 1100.

If information is received directly from diagnostic equipment 1250, any appropriate input may be used, such as parallel 10 circuit 1340 or serial 10 circuit 1342. Motion information received via the device 100 or via other diagnostic equipment 1250 may be analyzed using the mapping system 1347. In particular, the mapping system 1347 (e.g., control logic) may identify positions within the body of a patient and associate such positions with one or more electrodes where such electrodes may be capable of delivering stimulation energy to the heart, performing other actions or be associated with one or more sensors.

A communication interface 1345 optionally allows for wired or wireless communication with diagnostic equipment 1250 or other equipment (e.g., equipment to ablate or otherwise treat a patient). The communication interface 1345 may be a network interface connected to a network (e.g., intranet, Internet, etc.).

A map or model of cardiac information may be displayed using display 1214 based, in part, on 3-D heart information and optionally 3-D torso information that facilitates interpretation of information. Such 3-D information may be input via ports 1340, 1342, 1345 from, for example, a database, a 3-D imaging system, a 3-D location digitizing apparatus (e.g., stereotactic localization system with sensors and/or probes) capable of digitizing the 3-D location. While 3-D information and localization are mentioned, information may be provided with fewer dimensions (e.g., 1-D or 2-D). For example, where motion in one dimension is insignificant to one or more other dimensions, then fewer dimensions may be used, which can simplify procedures and reduce computing requirements of a programmer, an implantable device, etc. The programmer 1100 optionally records procedures and allows for playback (e.g., for subsequent review). For example, a heart map and all of the electrical activation data, mechanical activation data, VE data, etc., may be recorded for subsequent review, perhaps if an electrode needs to be repositioned or one or more other factors need to be changed (e.g., to achieve an optimal configuration). Electrodes may be lead based or non-lead based, for example, an implantable device may operate as an electrode and be self powered and controlled or be in a slave-master relationship with another implantable device (e.g., consider a satellite pacemaker, etc.). An implantable device may use one or more epicardial electrodes.

Once all pacing leads are mounted and all pacing devices are implanted (e.g., master pacemaker, satellite pacemaker, biventricular pacemaker), the various devices are optionally further programmed.

The telemetry subsystem 1322 may include its own separate CPU 1324 for coordinating the operations of the telemetry subsystem. In a dual CPU system, the main CPU 1302 of programmer communicates with telemetry subsystem CPU 1324 via internal bus 1304. Telemetry subsystem additionally includes a telemetry circuit 1326 connected to telemetry wand 1328, which, in turn, receives and transmits signals electromagnetically from a telemetry unit of the implanted device. The telemetry wand is placed over the chest of the patient near the implanted device 100 to permit reliable transmission of data between the telemetry wand and the implanted device.

Typically, at the beginning of the programming session, the external programming device 1100 controls the implanted device(s) 100 via appropriate signals generated by the telemetry wand to output all previously recorded patient and device diagnostic information. Patient diagnostic information may include, for example, motion information (e.g., cardiac, respiratory, etc.) recorded IEGM data and statistical patient data such as the percentage of paced versus sensed heartbeats. Device diagnostic data includes, for example, information representative of the operation of the implanted device such as lead impedances, battery voltages, battery recommended replacement time (RRT) information and the like.

Data retrieved from the implanted device(s) 100 can be stored by external programmer 1100 (e.g., within a random access memory (RAM) 1330, hard drive 1308, within a floppy diskette placed within floppy drive 1310). Additionally, or in the alternative, data may be permanently or semi-permanently stored within a compact disk (CD) or other digital media disk, if the overall system is configured with a drive for recording data onto digital media disks, such as a write once read many (WORM) drive. Where the programmer 1100 has a communication link to an external storage device or network storage device, then information may be stored in such a manner (e.g., on-site database, off-site database, etc.). The programmer 1100 optionally receives data from such storage devices.

A typical procedure may include transferring all patient and device diagnostic data stored in an implanted device 100 to the programmer 1100. The implanted device(s) 100 may be further controlled to transmit additional data in real time as it is detected by the implanted device(s) 100, such as additional motion information, IEGM data, lead impedance data, and the like. Additionally, or in the alternative, telemetry subsystem 1322 receives ECG signals from ECG leads 1332 via an ECG processing circuit 1334. As with data retrieved from the implanted device 100, signals received from the ECG leads are stored within one or more of the storage devices of the programmer 1100. Typically, ECG leads output analog electrical signals representative of the ECG. Accordingly, ECG circuit 1334 includes analog to digital conversion circuitry for converting the signals to digital data appropriate for further processing within programmer 1100. Depending upon the implementation, the ECG circuit 1343 may be configured to convert the analog signals into event record data for ease of processing along with the event record data retrieved from the implanted device. Typically, signals received from the ECG leads 1332 are received and processed in real time.

Thus, the programmer 1100 is configured to receive data from a variety of sources such as, but not limited to, the implanted device 100, the diagnostic equipment 1250 and directly or indirectly via external ECG leads (e.g., subsystem 1322 or external ECG system). The diagnostic equipment 1250 includes wired 1254 and/or wireless capabilities 1252 which optionally operate via a network that includes the programmer 1100 and the diagnostic equipment 1250 or data storage associated with the diagnostic equipment 1250.

Data retrieved from the implanted device(s) 100 typically includes parameters representative of the current programming state of the implanted devices. Under the control of the clinician, the external programmer displays the current programming parameters and permits the clinician to reprogram the parameters. To this end, the clinician enters appropriate commands via any of the aforementioned input devices and, under control of CPU 1302, the programming commands are converted to specific programming parameters for transmission to the implanted device 100 via telemetry wand 1328 to thereby reprogram the implanted device 100 or other devices, as appropriate.

Prior to reprogramming specific parameters, the clinician may control the external programmer 1100 to display any or all of the data retrieved from the implanted device 100, from the ECG leads 1332, including displays of ECGs, IEGMs, statistical patient information (e.g., via a database or other source), diagnostic equipment 1250, etc. Any or all of the information displayed by programmer may also be printed using a printer 1336.

A wide variety of parameters may be programmed by a clinician. In particular, for CRT, the AV delay and the VV delay of the implanted device(s) 100 are set to optimize cardiac function. In one example, the VV delay is first set to zero while the AV delay is adjusted to achieve the best possible cardiac function, optionally based on motion information. Then, VV delay may be adjusted to achieve still further enhancements in cardiac function.

Programmer 1100 optionally includes a modem to permit direct transmission of data to other programmers via the public switched telephone network (PSTN) or other interconnection line, such as a T1 line or fiber optic cable. Depending upon the implementation, the modem may be connected directly to internal bus 1304 may be connected to the internal bus via either a parallel port 1340 or a serial port 1342.

Other peripheral devices may be connected to the external programmer via the parallel port 1340, the serial port 1342, the communication interface 1345, etc. Although one of each is shown, a plurality of input output (IO) ports might be provided. A speaker 1344 is included for providing audible tones to the user, such as a warning beep in the event improper input is provided by the clinician. Telemetry subsystem 1322 additionally includes an analog output circuit 1346 for controlling the transmission of analog output signals, such as IEGM signals output to an ECG machine or chart recorder.

With the programmer 1100 configured as shown, a clinician or other user operating the external programmer is capable of retrieving, processing and displaying a wide range of information received from the ECG leads 1332, from the implanted device 100, the diagnostic equipment 1250, etc., and to reprogram the implanted device 100 or other implanted devices if needed. The descriptions provided herein with respect to FIG. 11 are intended merely to provide an overview of the operation of programmer and are not intended to describe in detail every feature of the hardware and software of the device and is not intended to provide an exhaustive list of the functions performed by the device 1100. Other devices, particularly computing devices, may be used.

### CONCLUSION

Although exemplary methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

What is claimed is:

1. A system comprising:
one or more processors;
memory; and
control logic to
acquire myocardial potential data associated with position information,
acquire myocardial electrical activation data associated with position information,
acquire myocardial position data with respect to time,
generate isopotential contours based on the potential data,
generate isochronal contours based on the electrical activation data,
generate isomotion contours based on the position data with respect to time, and
overlay the generated isopotential contours, isochronal contours and isomotion contours on a display to indicate a region of myocardial damage with respect to a map that comprises anatomical markers.

2. The system of claim 1 wherein the control logic to overlay comprises control logic to relatively weight the isopotential contours, the isochronal contours and the isomotion contours to increase or decrease their respective contribution to the overlay on the display to more accurately indicate a region of myocardial damage with respect to a map that comprises anatomical markers.

3. The system of claim 1 further comprising control logic to render adjustable controls to a display to individually weight the isopotential contours, the isochronal contours and the isomotion contours to increase or decrease their respective contribution to the overlay on the display to more accurately indicate a region of myocardial damage.

4. The system of claim 1 further comprising control logic to select only isopotential contours that comprise values less than a predetermined value to more accurately indicate a region of myocardial damage.

5. The system of claim 1 further comprising control logic to select only isochronal contours that comprise values greater than a predetermined value to more accurately indicate a region of myocardial damage.

6. The system of claim 1 further comprising control logic to select only isomotion contours that comprise values less than a predetermined value to more accurately indicate a region of myocardial damage.

7. The system of claim 1 further comprising control logic
to select only some of the contours based on one or more predetermined values and
to weight the selected contours to increase or decrease their respective contribution to the overlay on the display to more accurately indicate a region of myocardial damage.

8. The system of claim 1 further comprising control logic to indicate myocardial damage by outlining a scar region with respect to a map that comprises anatomical markers.

9. The system of claim 1 further comprising control logic to acquire fractionation data associated with position information, generate isofractionation metric contours based on the fractionation data and overlay the generated isofractionation metric contours and the generated isomotion contours on a display to indicate a region of myocardial damage with respect to a map that comprises anatomical markers wherein the fractionation data comprises data selected from a group of electrical fractionation data and mechanical fractionation data.

10. The system of claim 1 further comprising control logic to acquire dominant frequency data associated with position information, generate isofrequency contours based on the dominant frequency data and overlay the generated isofrequency contours and the generated isomotion contours on a display to indicate a region of myocardial damage with respect to a map that comprises anatomical markers wherein the dominant frequency data comprises data selected from a group of electrical dominant frequency data and mechanical dominant frequency data.

11. A method comprising:
mapping a first measure of cardiac performance on a map that comprises anatomical markers;
identifying a region on the map as including a myocardial scar;
selecting a second measure of cardiac performance;
mapping the second measure of cardiac performance on the map; and
narrowing the region on the map as including the scar.

12. The method of claim 11 wherein the first measure of cardiac performance comprises a measure selected from a group consisting of cardiac motion, cardiac potential and cardiac timing.

13. The method of claim 11 wherein the second measure of cardiac performance comprises a measure selected from a group consisting of cardiac motion, cardiac potential and cardiac timing.

14. The method of claim 11 wherein the narrowing comprises overlaying a contour for the first measure and a contour for the second measure.

15. The method of claim 14 wherein the overlaying defines an intersecting region.

16. The method of claim 15 wherein the intersecting region comprises a color caused by mixing a color associated with the contour for the first measure and a different color associated with the contour associated with the second measure.

17. The method of claim 11 further comprising
selecting a third measure of cardiac performance;
mapping the third measure of cardiac performance on the map; and
further narrowing the region on the map as including the scar.

18. The method of claim 11 wherein the mapping of the first measure creates a map that comprises isopotential contours.

19. The method of claim 11 wherein the mapping of the second measure creates a composite map that comprises isopotential and isochronal contours associated with activation of a heart.

20. The method of claim 11 wherein the mapping of the second measure creates a composite map that comprises isopotential and isomotion contours associated with activation of a heart.

21. The method of claim 11 wherein the mapping of the second measure creates a composite map that comprises isomotion and isochronal contours associated with activation of a heart.

22. The method of claim 11 further comprising determining a location for placement of an electrode in a patient's body based on the composite map.

23. The method of claim 11 wherein the first measure or the second measure comprises a measure selected from a group consisting of dominant frequency, fractionation, time to peak displacement, time to peak onset, time to peak slope, T wave morphology, Q wave morphology, ST segment and PR segment.

24. The method of claim 11 wherein the identifying occurs automatically responsive to the mapping of the first measure.

25. The method of claim 11 wherein the narrowing occurs automatically responsive to the mapping of the second measure.

26. The method of claim 11 further comprising providing one or more criterion associated with the first measure prior to identifying the region as including a myocardial scar.

27. The method of claim 11 further comprising providing one or more criterion associated with the second measure prior to narrowing the region as including the scar.

28. A system comprising:
one or more processors;
memory; and
control logic to
map a first measure of cardiac performance on a map that comprises anatomical markers,
identify a region on the map as including a myocardial scar,
select a second measure of cardiac performance,
map the second measure of cardiac performance on the map, and
narrow the region on the map as including the scar.

29. The system of claim 28 wherein the first measure of cardiac performance comprises a measure selected from a group consisting of cardiac motion, cardiac potential and cardiac timing.

30. The system of claim 28 wherein the second measure of cardiac performance comprises a measure selected from a group consisting of cardiac motion, cardiac potential and cardiac timing.

31. The system of claim 28 further comprising circuitry configured to acquire potentials from an electrode positioned in a current field and to determine a location for the electrode based on acquired potentials.

32. The system of claim 28 further comprising an input to receive image data for a heart and to map one or more anatomical markers based at least in part on received image data.

33. The system of claim 32 wherein the image data comprises image data selected from a group consisting of magnetic resonance image data, X-ray image data and ultrasound image data.

34. The system of claim 32 further comprising circuitry configured to acquire electrograms.

35. The system of claim 32 wherein the electrograms comprise intracardiac electrograms.
